(19) United States
(12) Patent Application Publication
(10) Pub. No.: US 2024/0412869 A1
(43) Pub. Date: Dec. 12, 2024

(54) Systems and Methods for Training and/or Using Representation Learning Neural Networks for Electromyographic Data

(71) Applicant: Emory University, Atlanta, GA (US)

(72) Inventors: Chethan Pandarinath, Decatur, GA (US); Lahiru Neth Wimalasena, Atlanta, GA (US); Mohammadreza Keshtkaran, Atlanta, GA (US)

(21) Appl. No.: 18/701,602

(22) PCT Filed: Oct. 13, 2022
(86) PCT No.: PCT/US2022/046615

§ 371 Date: Apr. 15, 2024

Related U.S. Application Data
(60) Provisional application No. 63/255,317, filed on Oct. 13, 2021.

(51) Int. Cl. G16H 50/20; G06F 3/01

(52) U.S. Cl. CPC G16H 50/20; G06F 3/015

(57) ABSTRACT

Systems and methods that use data augmentation during the training of representation learning networks on neuromuscular data (e.g., EMG) with reconstruction cost. The method may include training network(s) on neuromuscular data received from channel(s) of neuromuscular sensor(s). The training may include randomly generating a first augment and a different, second augment for each channel. The training may include augmenting the data of each channel by applying the first and second augments to the data to generate first and second augmented data. The training may include processing at least the first augmented data through at least a first representation learning neural network to determine a first latent representation of one or more neuromuscular activation state variables. The training may include determining a reconstruction cost using the first latent representation and the second augmented data. The training may include updating the first representation learning neural network based on the reconstruction cost.

### CROSS-REFERENCE TO RELATED APPLICATIONS

This application claims the benefit of U.S. Provisional Application No. 63/255,317 filed Oct. 13, 2021. The entirety of this application is hereby incorporated by reference for all purposes.

### STATEMENT REGARDING FEDERALLY SPONSORED RESEARCH OR DEVELOPMENT

This invention was made with government support under U.S. Pat. No. 1,835,364 awarded by the National Science Foundation, HR0011-19-9-0045 awarded by DARPA, and HD073945 awarded by the National Institutes of Health. The government has certain rights in the invention.

### BACKGROUND

Neuromuscular data, such as electromyographic (EMG) data, recorded by neuromuscular sensors, such as EMG sensors, can be used to assess neuromuscular dysfunction in patients, such as those with motor control disorders, as well as to control devices, such as robotic hardware, prostheses, prosthetics, human-computer interface devices (e.g., mouse, keyboard, touch screen, game controller, etc.), etc.

Currently available techniques have difficulty of estimating muscle activation from EMG data. For example, training representation learning networks on EMG data can be susceptible to finding trivial solutions (i.e., output of the representation learning neural networks is similar to input, and the underlying structure is not modeled). This can be due to the presence of correlated noise, or other signals that are highly correlated across EMG data but are undesirable to be preserved in the output of the network. As a result, these networks can provide an inaccurate measurement of muscle activation.

### SUMMARY

Thus, there is a need for training neural networks, such as representation learning networks, on EMG data that produce models that avoid trivial solutions.

Techniques disclosed herein relate to using data augmentation during the training of representation learning neural networks on neuromuscular data to produce models that avoid trivial solutions, therefore resulting in better performing models that yield better de-noising of data and enable better prediction of variables that are correlated with the data. This can result in more accurate measurements of muscle activation which can be useful for downstream applications, for example, for controlling devices.

The disclosed embodiments may include computer-implemented systems and methods, as well as non-transitory computer-readable media, for training one or more networks on neuromuscular data (e.g., EMG) using data augments. In some examples, the method may include receiving, via one or more channels of one or more neuromuscular sensors, neuromuscular data for a preset window from each channel. The method may also include training at least one or more networks using the first augmented data and the second augmented data. The one or more networks may include one or more representation learning neural networks. In some examples, the training the one or more representation learning neural networks may include randomly generating a first augment and a second augment different from the first augment for each channel. The training the one or more representation learning neural networks may also include augmenting the neuromuscular data of each channel by applying the first augment and the second augment to the neuromuscular data of respective channel to generate a first augmented data and a second augmented data for the respective channel. The training the one or more representation learning neural networks may include processing at least the first augmented data for each channel through at least a first representation learning neural network to determine a first latent representation of one or more neuromuscular activation state variables for the respective channel. The training the one or more representation learning neural networks may include determining a reconstruction cost using the first latent representation and the second augmented data for each channel. The training the one or more representation learning neural networks may include updating the first representation learning neural network based on the reconstruction cost.

In some examples, the system may include one or more processors and one or more neuromuscular sensors coupled to the processor. Each neuromuscular sensor may include one or more channels. The system may also include a memory having stored thereon computer-executable instructions which are executable by the one or more processors to cause the computing system to perform at least receiving, via one or more channels of one or more neuromuscular sensors, neuromuscular data for a preset window from each channel. The one or more processors may be further configured to cause the computing system to perform at least training at least one or more networks using the first augmented data and the second augmented data. The one or more networks may include one or more representation learning neural networks. The training the one or more representation learning neural networks may include randomly generating a first augment and a second augment different from the first augment for each channel. The training the one or more representation learning neural networks may include augmenting the neuromuscular data of each channel by applying the first augment and the second augment to the neuromuscular data of respective channel to generate a first augmented data and a second augmented data for the respective channel. The training the one or more representation learning neural networks may include processing at least the first augmented data for each channel through at least a first representation learning neural network to determine a first latent representation of one or more neuromuscular activation state variables for the respective channel. The training the one or more representation learning neural networks may include determining a reconstruction cost using the first latent representation and the second augmented data for each channel. The training the one or more representation learning neural networks may include updating the first representation learning neural network based on the reconstruction cost.

In some examples, the non-transitory computer-readable storage medium may comprise one or more computer-executable instructions, that when executed by one or more processors of a computing device, may cause the computing device to perform at least receiving, via one or more channels of one or more neuromuscular sensors, neuromuscular data for a preset window from each channel. The one or more processors may be further configured to cause the computing system to perform at least training at least one or more networks using the first augmented data and the second augmented data. The one or more networks may include one or more representation learning neural networks. The training the one or more representation learning neural networks may include randomly generating a first augment and a second augment different from the first augment for each channel. The training the one or more representation learning neural networks may include augmenting the neuromuscular data of each channel by applying the first augment and the second augment to the neuromuscular data of respective channel to generate a first augmented data and a second augmented data for the respective channel. The training the one or more representation learning neural networks may include processing at least the first augmented data for each channel through at least a first representation learning neural network to determine a first latent representation of one or more neuromuscular activation state variables for the respective channel. The training the one or more representation learning neural networks may include determining a reconstruction cost using the first latent representation and the second augmented data for each channel. The training the one or more representation learning neural networks may include updating the first representation learning neural network based on the reconstruction cost.

In some examples, each augment may correspond to a temporal shift. In some examples, each temporal shift may have an integer value.

In some examples, the augmenting the neuromuscular data for each channel may include moving the neuromuscular data forward or backward temporally within the preset window based on the integer value of the respective temporal shift.

In some examples, the one or more networks may include a second representation learning network. In some examples, the method may further include and/or the one or more processors may be further configured to cause the computing system to perform at least processing the second augmented data for each channel through a second representation learning neural network to determine a second latent representation of one or more neuromuscular activation state variables for the preset window of time. The reconstruction cost may be determined using the first latent representation and the second latent representation. The method may further include and/or the one or more processors may be further configured to cause the computing system to perform at least updating the first representation learning neural network and/or the second representation learning neural network based on the reconstruction cost.

In some examples, the one or more networks may include a projector network. In some examples, the method may further include and/or the one or more processors may be further configured to cause the computing system to perform at least processing the first latent representation of one or more neuromuscular activation state variables for the preset window of time through a projector network to generate a first projected latent representation. The reconstruction cost may be determined between the first projected latent representation and the second latent representation. The method may further include and/or the one or more processors may be further configured to cause the computing system to perform at least updating the first representation learning neural network and/or the second representation learning neural network based on the reconstruction cost.

In some examples, the only first representation learning neural network may be updated based on the reconstruction cost. For example, the second representation learning neural network may be updated using a moving average of the parameters of the first representation learning neural network.

In some examples, the one or more representation learning neural networks may include one or more of autoencoder neural networks, one or more of transformer neural networks and/or one or more of linear transformation networks.

In some examples, each augment may include a temporal value, a magnitude value, a randomly generated temporal order, and/or a randomly generated binary mask.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, the emphasis being placed upon illustrating the principles of the disclosure.

FIG. 1 illustrates an example of a system environment for determining one or more neuromuscular activation states using one or more trained representation learning neural networks according to embodiments.

FIG. 2 is a flow chart illustrating an example of a method of determining one or more neuromuscular activation states by applying one or more trained networks, including at least one trained representation learning neural network, on neuromuscular data according to embodiments.

FIG. 3 is a flow chart illustrating an example of a method of training representation learning neural network(s) on neuromuscular data according to embodiments.

FIG. 4A is an illustrative example of neuromuscular data received from one or more channels of one or more neuromuscular sensors for training the representation learning neural network(s); FIG. 4B is an isolated view of the data from one of the channels shown in FIG. 4A.

FIG. 5 is an illustrative example of augmenting the data by applying a randomly generated temporal shift to the neuromuscular data of FIG. 4A for one of the channels according to embodiments.

FIG. 6 is an illustrative example of augmenting the data by applying a randomly generated temporal shift to the neuromuscular data of FIG. 4A for another one of the channels according to embodiments.

FIG. 7 is a flow chart illustrating an example of a method of training a representation learning neural network on neuromuscular data according to embodiments.

FIG. 8 is an illustrative example of training a representation learning neural network according to FIG. 7 using the augmented neuromuscular data of FIGS. 5 and 6 according to embodiments.

FIG. 9 is a flow chart illustrating another example of a method of training representation learning neural networks on neuromuscular data according to embodiments.

FIG. 10 is an illustrative example of training representation learning neural networks according to FIG. 9 using the augmented neuromuscular data of FIGS. 5 and 6 according to embodiments.

FIG. 11 is an illustrative example of a model schematic modeling dynamics underlying EMG activity with AutoLFADS according to embodiments used in the Example. FIG. 11 shows a modified AutoLFADS to accommodate the statistics of EMG activity using a gamma observation model rather than Poisson. m is a small window from a continuous recording of rectified EMGs from a group of muscles. m[t] is a vector representing the multi-muscle rectified EMG at each timepoint. \hat{m}[t] is an estimate of muscle activation from the EMG activation envelopes m[t]. Our approach models \hat{m}[t] as the output of a nonlinear dynamical system with shared inputs to the muscles. The Generator RNN is trained to model the state update function ƒ, which captures the spatial and temporal regularities that underlie the coordinated activation of multiple muscles. The other RNNs provide the Generator with an estimate of initial state s[0] and a time-varying input u[t], allowing the dynamical system to evolve in time to generate s[t]. \hat{m}[t] is taken as a nonlinear transformation of s[t]. All of the model's parameters are updated via backpropagation of the gamma log-likelihood of observing the input rectified EMG data.

FIGS. 12A-C show an illustrative example of applying AutoLFADS to rat locomotion according to embodiments used in the Example. FIG. 12A shows a schematic of rat locomotion Lower left trace indicates “stance” and “swing” phases during a single stride, swing labeled below in orange. On the right are joint angular velocities (θ′) and accelerations (θ″) for hip, knee, and ankle over three consecutive strides. FIG. 12B shows an example of an AutoLFADS output overlaid on rectified, unfiltered EMG for three muscles in the rat hindlimb (RF: rectus femoris, GA: gastrocnemius, TA: tibialis anterior). FIG. 12C shows the top 3 principal components of the AutoLFADS output for individual strides, colored by phase of locomotion (total variance explained: 92%). Arrows indicate the direction of the state evolution over time. Foot strike events are labeled with white dots.

FIG. 13 shows an illustrative example of AutoLFADS filtering that is adapted to preserve behaviorally relevant, high-frequency EMG features according to embodiments used in the Example. Approximated frequency responses of a 20 Hz lowpass filter and AutoLFADS applied to EMG for 4 muscles in the swing (solid) and stance (dashed) phases (GS: gluteus superficialis, RF: rectus femoris, IL: iliopsoas, GA: gastrocnemius). Shaded area represents SEM of estimated frequency responses from single stride estimates. Estimated frequency responses from single strides were highly consistent, resulting in small SEM. Gray vertical line at 20 Hz signifies low pass filter cutoff.

FIGS. 14A-C show illustrative examples of AutoLFADS muscle activation estimates determined in the Example that are more predictive of joint angular acceleration than other filtering approaches. FIG. 14A shows a visualization of single trial joint angular accelerations (θ″) for hip, knee, and ankle joint angular kinematic parameters. Trials were aligned to the time of foot off using an optimal shift alignment method, then grouped and colored based on the magnitude of the hip angular acceleration at a specific time (indicated by dashed line). FIG. 14B shows a joint angular acceleration decoding performance for AutoLFADS, Bayesian filtering approaches, and multiple low-pass filter cutoff frequencies, quantified using variance accounted for (VAF). Each bar represents the mean+/−SEM across cross-validated prediction VAF values for a given joint. FIG. 14C shows a visualization of single trial muscle activation estimates for AutoLFADS, 20 Hz low-pass filter (LF θ″ opt), Bayesian filtering with hyperparameters optimized to maximize prediction of θ″ (BF θ″ opt), and Bayesian filtering using the same hyperparameters as Sanger, 2007. Alignment and coloring of single trials are the same as FIG. 14A. Arrows highlight visual features that differentiate muscle activation estimates from tested methods.

FIGS. 15A-C show illustrative examples of applying AutoLFADS to isometric wrist tasks according to embodiments used in the Example. FIG. 15A shows a schematic of monkey isometric wrist task. Force and dF/dt are shown for three consecutive successful trials. The target acquisition phases are indicated by arrows corresponding to the target directions. Black lines denote target onset. Grey intervals denote return to and hold within the central target. FIG. 15B shows an example of AutoLFADS output overlaid on rectified EMG for four forearm muscles that have a moment about the wrist (EDC: extensor digitorum communis, ECU: extensor carpi ulnaris, FCU: flexor carpi ulnaris, FDS: flexor digitorum superficialis). FIG. 15C shows top three principal components of AutoLFADS output for four conditions, colored by target hold location (total variance explained: 88%).

FIGS. 16A-F show illustrative examples of AutoLFADS that enhances high-frequency coherence with dF/dt. FIG. 16A shows single trial oscillations that were captured in the x component of recorded force (and more clearly in in dFₓ/dt) for the flexion target direction. FIG. 16B shows corresponding oscillations that were faintly visible in the rectified EMG. FIG. 16C shows single trial muscle activation estimates from different approaches. FIG. 16D shows single trial oscillations of dFₓ/dt for three of the eight conditions, specifically to targets in the upper/right quadrant. Thick traces: trial average. FIG. 16E shows single trial oscillations were also visible in AutoLFADS output across the wrist and digit flexor muscles (FCR: flexor carpi radialis, FDP: flexor digitorum profundus, FDS: flexor digitorum superficialis) (f) Magnitude-squared coherence computed between single trial EMG for a given muscle and dFₓ/dt) and averaged across trials. Each line represents the mean+/−SEM coherence to dFₓ/dt) for a given muscle activation estimate. Coherence was compared between AutoLFADS, If 20 EMG, Bayesian filtering with hyperparameters optimized to maximize prediction of dF/dt, and Bayesian filtering using the same hyperparameters as Sanger, 2007.

FIG. 17 show scatter plot comparing accuracy of the prediction of motor cortical activity from EMG using different filtering approaches and AutoLFADS used in eh Example. Each dot represents prediction accuracy in terms of VAF for a single M1 channel. FIG. 17 shows that AutoLFADS can improve correlation with motor cortical (M1) activity.

FIG. 18 is a simplified block diagram of an example of a computing system for implementing certain embodiments disclosed herein.

### DESCRIPTION OF THE EMBODIMENTS

In the following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The disclosed embodiments generally relate to methods and systems that use data augmentation (e.g., temporal shift), a self-supervised learning approach, during the training of the representation learning networks (also referred to as “models”) on neuromuscular data, such as electromyography (EMG) data, with reconstruction cost. The disclosed methods and systems use data augmentation to disrupt representation learning models from learning spurious correlations that can exist in multi-muscle neuromuscular (EMG) recordings (and other high-density neural recording modalities) so that automated optimization paradigms for model training better learn the underlying latent structure in the data that is useful for downstream applications (e.g., controlling prostheses, computer interfaces, etc.). The disclosed methods and systems can therefore prevent overfitting, such as to high-magnitude events that can occur simultaneously across multiple recording channels of either a single or multiple neuromuscular sensor(s). Thus, the disclosed methods and systems can improve the estimation of the latent representation of one more neuromuscular activation state variables and therefore improve the utility of such representations for downstream applications.

In some examples, the disclosed techniques randomly manipulate neuromuscular data of each recording channel of either a single or multiple neuromuscular sensor(s) using a randomly generated augment (e.g., temporal shift) when inputting the neuromuscular data to a representation learning neural network during training. In some examples, the model input and the data used to compute a reconstruction cost can be manipulated differently to prevent the representation learning neural network from overfitting by drawing a direct correspondence between specific values of the input and output. By improving the training of representation learning neural networks on neuromuscular data (e.g., EMG data), the disclosed method and systems can improve the estimation of the latent representation of one or more neuromuscular activation state variables. Therefore, the disclosed methods and systems can improve the utility of such representations for downstream applications, such as controlling target devices.

For example, the disclosed methods and systems can enable representation learning neural networks to be trained to extract latent signals from neuromuscular data (e.g., EMG data) that can be used to control devices, such as human-computer interface-controlled device and/or muscle-controlled prosthetics or assistive devices. The networks trained according to the disclosure can improve inference of muscle activation from monitored neuromuscular data thereby yielding better prediction of behavior (e.g., force, kinematics, etc.) and brain activity (i.e., motor cortical activity). Thus, the user experience with neuromuscular-controlled devices can be improved.

The disclosed methods and system randomly generate an augment to manipulate the training neuromuscular data for each window to generate augmented data. An augment generally refers to a randomly generated change that results in a temporal and/or magnitude modification in the training neuromuscular data to generate additional training data (augmented data) when the augment is applied. For example, an augment may include but is not limited to a randomly generated temporal shift value that refers to an integer value based on which the data is moved forward or backward; a randomly generated magnitude value that refers to a value based on which the magnitude of data is modified; a randomly generated binary mask based on which the datapoints at specific time samples are replaced with a null value (e.g., zero); a randomly generated temporal order based on which the order or sequence of datapoints within the window is modified; among others; or a combination thereof.

While examples of the disclosure may be specific to processing electromyographic (EMG) data, it will be understood that these examples are nonlimiting and that the disclosed methods and systems may be used to train on other types of neuromuscular data, such as those disclosed therein. It will also be understood that while examples of the disclosure may be specific to augmenting the neuromuscular data using a temporal shift, these examples are nonlimiting and that the disclosed methods and systems can use other randomly generated augments, such as those disclosed herein, to augment (i.e., manipulate) the neuromuscular data.

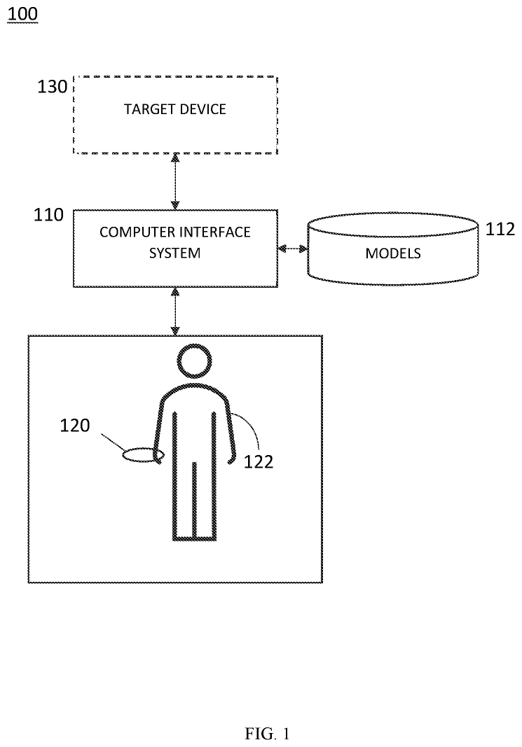

FIG. 1 depicts an example of a system environment 100 that can include a neuromuscular data analysis system 110 that is configured to decode neuromuscular data of a user 122 received from one or more sensors 120 using a stored trained (or retrained) networks (also referred to as “models”) 112 to a command, corresponding to a neuromuscular activation state, for controlling a target device 130 according to embodiments. In some examples, the neuromuscular data analysis system 110 may alternatively and/or additionally be used to train the one or more networks 112 to accurately generate latent representations, corresponding to a neuromuscular activation state, that can be decoded to command the target device 130.

In some examples, the neuromuscular data may refer to signals arising from neuromuscular activity (e.g., neural activation of spinal motor neurons that innervate a muscle, muscle activation, muscle contraction, or a combination thereof) in muscles of a human body recorded by the sensor(s) 120. In some examples, the neuromuscular data may include but is not limited to EMG data, mechanomyography (MMG) data, sonomyography (SMG) data, among others, and/or any combination thereof. For example, the one or more sensors 120 may include but is not limited to EMG sensors, MMG sensors, SMG sensors, among others, and/or any combination thereof. By way of example, the one or more sensors 120 may be a multi-channel EMG sensor system, for example, disposed on a wearable device and/or implantable array. In some examples, the one or more sensors 120 may be a single-channel multi-sensor device, for example, disposed on a wearable device and/or implantable array.

In some embodiments, the one or more sensors 120 may be operatively connected (invasively and/or non-invasively) to the user 122 and configured to generate neuromuscular data responsive to the user's muscular activity. In some embodiments, the one or more sensors 120 may be any currently available or later developed invasive sensor(s), non-invasive(s) sensors, or any combination thereof. In some examples, the one or more sensors 120 may be operatively connected with one or more muscles and/or body segment of the user. For example, the sensor(s) 120 may include an microelectrode array disposed in a muscle and/or body segment of the user configured to obtain neuromuscular data; a wearable microelectrode array (e.g., integrated into a headband, hat, eyeglasses, headset, other head-worn article, wristband, suite, among other, or any combination thereof) that can be disposed outside of the user and on a respective muscle and configured to obtain neuromuscular data noninvasively, such as a human machine/computer interface; among others; or any combination thereof.

In some embodiments, the one or more stored trained networks 112 may include one or more trained representation learning neural network(s), projector network(s), among others, or a combination thereof. For example, the trained representation learning neural network(s) may include but is not limited to autoencoder neural network(s), transformer neural network(s), linear transformation networks, other latent variable models that capture underlying structure from observed data, or any combination thereof. In some examples, the stored trained representation learning neural network may be an adapted latent factor analysis via dynamical systems (AutoLFADs) model as described in the Example below. In some embodiments, the trained representation learning neural network(s) may include an encoder and a decoder. By way of another example, the projector network may include but is not limited to a linear transformation network and/or other neural network, configured to map the latent representations of a first representation learning neural network to match the latent representations from a second representation learning neural network. For example, please see B. Grill, F. Strub, F. Altch e, C. Tallec, P. H. Richemond, E. Buchatskaya, C. Doersch, B. A. Pires, Z. D. Guo, M. G. Azar, et al., 2020. “Bootstrap your own latent: A new approach to self-supervised learning,” arXiv preprint arXiv:2006.07733, which is incorporated by reference in its entirety.

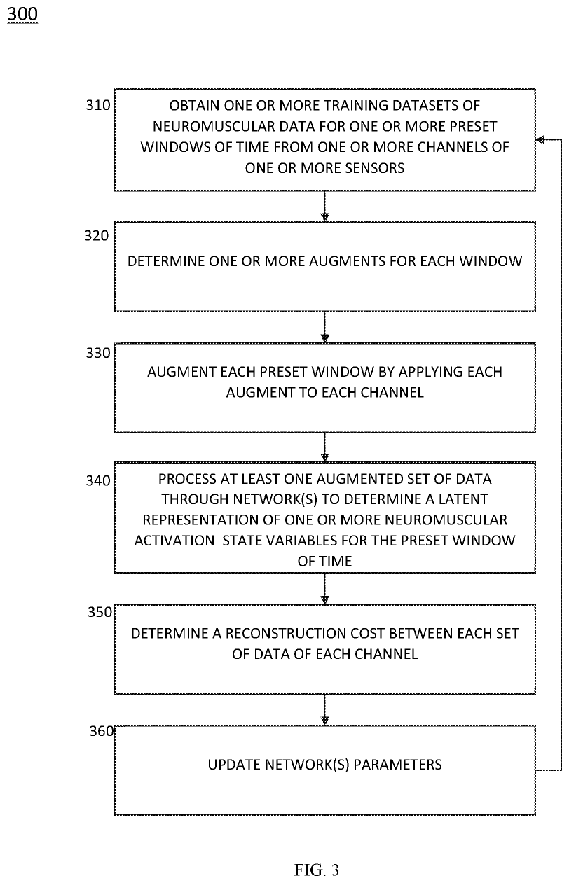

In some embodiments, the one or more networks 112 may be trained using data augmentation. In some examples, a randomly generated augment may be generated to augment the training data. The augmented training data may be used to train the networks, for example, based on the reconstruction cost according to embodiments. FIGS. 3-10 show examples of training network(s) using data augmentation (e.g., temporal shift) according to embodiments.

In some embodiments, the neuromuscular data analysis system 110 may include any computing or data processing device consistent with the disclosed embodiments. In some embodiments, the system 110 may incorporate the functionalities associated with a personal computer, a laptop computer, a tablet computer, a notebook computer, a hand-held computer, a personal digital assistant, a portable navigation device, a mobile phone, an embedded device, a smartphone, and/or any additional or alternate computing device/system. The system 110 may transmit and receive data across a communication network. In some embodiments, the neural data analysis system 110 may be envisioned as a human-computer interface controlling the target device 130 according to the decoded neural data.

In some embodiments, the target device 130 may include but is not limited to a robotic arm or device, full limb prosthesis, partial limb prosthesis, neuroprosthesis or functional electrical stimulation (FES) device that actuates a paralyzed limb, an orthotic device, a remote hands free device (e.g., a robot or an unmanned aerial vehicle), a motor vehicle, interface devices (e.g., keyboard, pointing devices, etc.) that are configured to interact with a content displayed on a computer and/or portable electronic display (e.g., head-mounted display), other human-interface controlled devices, other electronic devices, among others, or a combination thereof.

In some embodiments, the neuromuscular analysis system 110 may be configured to communicate with the one or more sensors 120, the target device 130, another programming or computing device via a wired or wireless connection using any of a variety of local wireless communication techniques, such as RF communication according to the 802.11 or Bluetooth specification sets, infrared (IR) communication according to the IRDA specification set, or other standard or proprietary telemetry protocols. The system 110 may also communicate with other programming or computing devices, such as the target device 130, or one or more sensors 120, via exchange of removable media, such as memory cards. In other embodiments, the one or more sensors 120 and/or the target device 130 may incorporate the functionalities discussed and associated with the system 110.

Although the systems/devices of the environment 100 are shown as being directly connected, the system 110 may be indirectly connected to one or more of the other systems/devices of the environment 100. In some embodiments, the system 110 may be only directly connected to one or more of the other systems/devices of the environment 100.

It is also to be understood that the environment 100 may omit any of the devices illustrated and/or may include additional systems and/or devices not shown. It is also to be understood that more than one device and/or system may be part of the environment 100 although one of each device and/or system is illustrated in the environment 100. It is further to be understood that each of the plurality of devices and/or systems may be different or may be the same. For example, one or more of the devices may be hosted at any of the other devices.

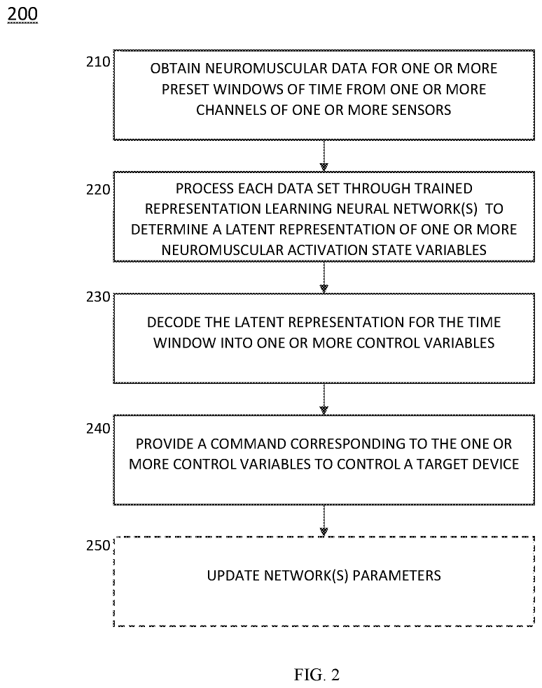

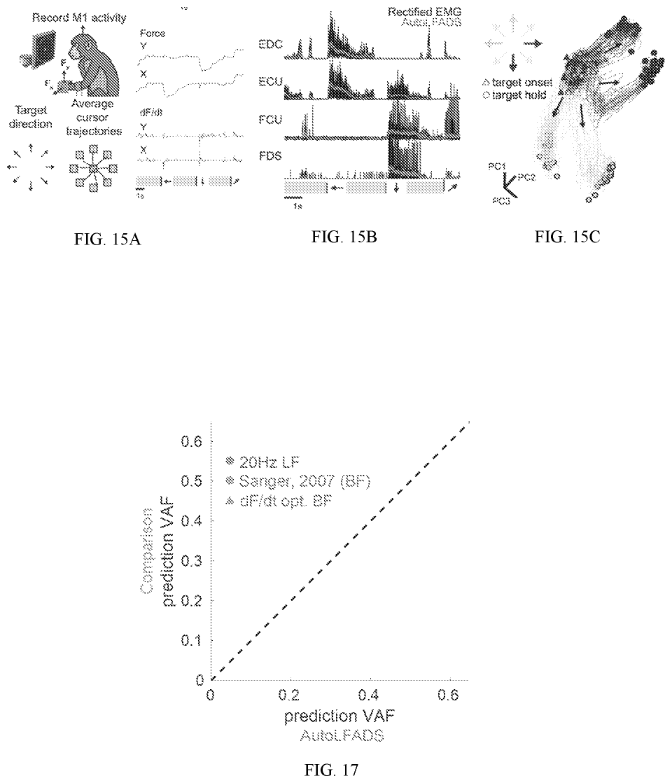

FIGS. 2, 3 and 7-10 show flow charts according to embodiments. Operations described in flow charts 200, 300, and 700-1000 may be performed by a computing system, such as the system 110 described above with respect to FIG. 1 or a computing system described below with respect to FIG. 15. Although the flow charts 200, 300, and 700-1000 may describe the operations as a sequential process, in various embodiments, some of the operations may be performed in parallel or concurrently. In addition, the order of the operations may be rearranged. An operation may have additional steps not shown in the figure. In some embodiments, some operations may be optional. Embodiments of the method/architecture may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

FIG. 2 shows a flow chart 200 illustrating an example of a method of decoding neuromuscular data into control signals for controlling the target device using the trained neural networks according to embodiments. Operations in flow chart 200 may begin at block 210, the system 110 may receive neuromuscular data associated with a user for one or more preset windows of time from each channel of the one or more sensors 120. For example, the neuromuscular data may be received from a multi-channel electrode disposed intra-muscularly or on a surface of a body (over the target muscles) of the user 122. In some examples, the neuromuscular data may be received from multiple single-channel electrodes disposed intra-muscularly or on a surface of a body (over the target muscles) of the user 122.

In some embodiments, at block 220, the system 110 may process the received neuromuscular data through the trained network(s) to determine one or more neuromuscular activation state variables for the period of time. In some examples, the trained networks may include one or more representation neural networks. For example, the one or more neuromuscular activation state variables (also referred to as “latent variables”) may include but is not limited to the magnitude of a one or more muscle's activation signals, among others, or any combination thereof. FIGS. 3 and 7-10 show examples of training network(s) according to embodiments.

In some embodiments, at block 240, the system 100 may decode the one or more neuromuscular activation state variables into one or more control variables corresponding to that neuromuscular activation state(s). For example, the neuromuscular activation state may relate to the muscular activation of one or more groups of coordinated muscles.

In some embodiments, at block 240, the system 110 may provide a command or cause initiation of the command by the target device 130 corresponding to the one or more control variables determined at block 230. For example, the system 110 may cause movement of a robotic or prosthetic limb, movement of a peripheral device (e.g., a computer mouse, vehicle, etc.), action by a communication device, interaction with content on a display, among others, or any combination thereof.

In some embodiments, at block 250, the system 110 may optionally update the trained network(s), for example, as described in and discussed with respect FIGS. 3 and 7-10.

FIG. 3 shows a flowchart 300 illustrating examples of methods for training the network(s) using randomly generated augments according to embodiments. In some examples, at block 310, one or more training datasets of neuromuscular data for one or more preset windows of time (also referred to as “time window”) from one or more channels of one or more sensors (e.g., single-channel and/or multi-channel EMG sensor(s)) may be obtained for a subject. In some examples, the training dataset for each channel of one or more sensors may include one or more windows of time. In some examples, each window of time may include a set number of discrete timesteps (also referred to as “bins”). In some examples, each window of time may include a preset number of subset windows of time. Each subset window may include a preset number of timesteps.

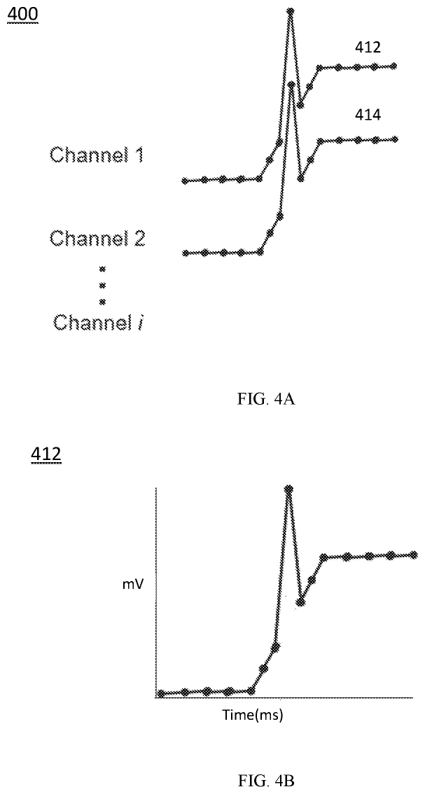

FIG. 4A shows an example 400 of a training set of neuromuscular data received for a preset window from one or more channels of one or more sensors. The neuromuscular data may be received from each channel of a multi-channel sensor and/or each channel of multiple single-channel sensor(s). In this example, for the preset window, data 412 was received for Channel 1 and data 414 was received for Channel 2. FIG. 4B shows an isolated view of the data 412. For example, Channel 1, 2, . . . i may correspond to different channels of the single multi-channel neuromuscular sensor (e.g., EMG sensor) or may correspond to channels of different single-channel neuromuscular sensors (e.g., Channel 1 corresponds to single-channel neuromuscular Sensor 1, Channel 2 corresponds to single-channel muscular Sensor 2, etc.). It will be understood that data could be received from a multi-channel neuromuscular sensor that includes more than two channels (e.g., three channels, four channels, more than four channels, i number of channels, etc.); data could be received from more than two single-channel neuromuscular sensors (e.g., three sensors, four sensors, more than four sensors, i number of channels, etc.). In some examples, the training data could be received for each channel of the one or more neuromuscular sensors for that preset window of time.

As shown, the window of time may include a set number of discrete timesteps (also referred to as “bins”). In FIGS. 4A and 4B, each dot corresponds to a timestep (also referred to as “bin”) within the preset window. Although not shown, each preset window may include a number of subset windows. For example, each subset window may include a preset and equal number of time timesteps (i.e., dots).

Next at block 320, for each channel, one or more augments may be randomly generated for the preset window. For example, an augment may include but is not limited to a randomly generated temporal shift value that refers to an integer value based on which the data is moved forward or backward; a randomly generated magnitude value that refers to a value based on which the magnitude of data is modified; a randomly generated binary mask (also referred to as “masking”) based on which the datapoints at specific time samples are replaced with a null value (e.g., zero); a randomly generated temporal order based on which the order or sequence of datapoints within the window is modified; among others; or a combination thereof. In some examples, two different augments may be generated for the data of each channel. In some examples, more than two augments may be generated for the data of each channel.

In some examples, if the augment includes a temporal shift, each temporal shift may be a value randomly generated by sampling integer values from a distribution (e.g., normal distribution). In some examples, the randomly generated value may be bounded by a maximum shift value. In some examples, the maximum shift value may be preset. The temporal shift may be a negative or a positive integer value. In some examples, two different temporal shifts may be randomly generated for each channel. By way of example, a first temporal shift may be generated by randomly sampling an integer value from a distribution (e.g., normal distribution) and a second temporal shift may be generated by randomly sampling another integer value from the same distribution.

For example, if the augment includes a randomly generated magnitude value, each magnitude value may be randomly generated by sampling floating point values from a distribution (e.g., normal distribution). In some examples, the maximum randomly generated magnitude value may be preset. The magnitude value may be a negative or positive floating point value. In some examples, a randomly generated magnitude value may be generated for every time sample for each channel.

For example, if the augment includes a masking, randomly selected time samples may have the corresponding datapoints (data corresponding to a timestep for a given channel) set to a null value (e.g., zero), effectively preventing the representation learning neural network from using the information from these timepoints. The masking may also operate at the level of channels, where all time samples of a randomly selected channel may be masked. By way of example, if one channel is randomly selected, all time samples for the window for that channel could be masked.

By way of another example, if the augment includes a randomly generated temporal order, the window subsets may be rearranged according to a randomly generated sequence or order so that the corresponding datapoints (data corresponding to timestep) within the window subset may be arranged according to the randomly generated sequence or order.

Next, at block 330, the neuromuscular data for each channel may be augmented by applying each augment to generate more than one augmented data for each channel. For example, if two different augments were generated for each channel, a first data may be applied to the data of that channel to generate a first augmented data (x->x′) and a second augment may be applied to the data of that channel to generate a second augmented data (x->x″).

By way of example, if the augment includes a temporal shift, applying each augment may cause the data to move a number of timesteps (bins) forward or backward corresponding to the temporal shift. For example, the data of each preset window for each channel may be augmented using a first temporal shift to generate a first augmented data and using a second, different temporal shift to generate a second augmented data.

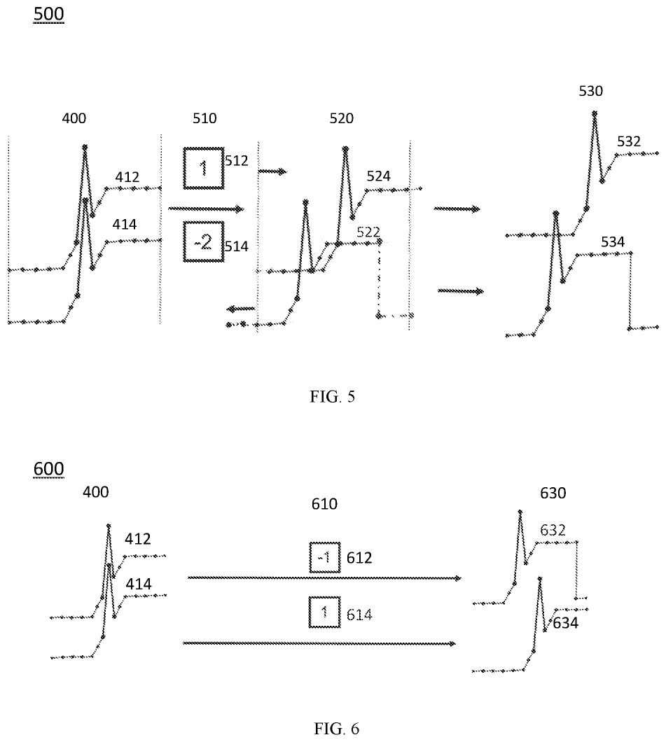

FIGS. 5 and 6 show examples of augmenting the data 400 for the Channel 1 (412) and the Channel 2 (414) by applying randomly generated temporal shifts to generate augmented data 530, respectively. FIG. 5 shows an example 500 of generating first augmented data 530 for the data 400 for the Channels using randomly generated temporal shifts 510. In this example, the randomly generated temporal shift 512 for Channel 1 corresponds to “1” and the randomly generated temporal shift 514 for Channel 2 corresponds to “−2”. By way of example, the maximum value for the temporal shift could be “6” so the randomly generated temporal shift for each channel can be any integer value greater than or equal to “−6” and less than or equal to “6”.

When the randomly generated temporal shifts 510 are applied (520), data for the window for each channel can be shifted a number of bins forward or backward based on the generated temporal shift (510). For example, as shown, the data 412 for Channel 1 can be shifted 1 timestep forward (522) corresponding the temporal shift 512. By way of example, as shown, the data 414 can be shifted 2 timesteps backward (524) corresponding to the temporal shift 514.

In some examples, the data for each channel may be further processed based on the shift. By way of example, as a result of the temporal shift, any data that is moved out of the data window can be trimmed or removed (shown as dashed line), and any gaps in data can be padded (shown in dot-dashed line) in FIG. 5. The shifted and processed data in 520 may result in the first augmented data 530 for each channel. As shown, the first augmented data 532 for Channel 1 and the first augmented data 534 for Channel 2 has been shifted from the original data 400 based on the temporal shift 510.

FIG. 6 shows an example 600 of generating second augmented data 630 for the data 400 for Channels 1 and 2 using randomly generated temporal shift 610. In this example, the randomly generated temporal shift 612 for the Channel 1 corresponds to “−1” and the randomly generated temporal shift 614 for the Channel 2 corresponds to “1”. Like the example 500, when the randomly generated temporal shifts 610 are applied, each channel data for the window can be shifted a number of bins forward or backward based on the integer value. For example, the data 412 for Channel 1 can be shifted 1 timestep backward corresponding the temporal shift 612 and the data 414 can be shifted 1 timestep forward corresponding to the temporal shift 614. Like the example 500, the data may be further processed to trim and/or pad the shifted data. The shifted and processed data in may result in the second augmented data 630 for each channel. As shown, the second augmented data 632 for Channel 1 and the second augmented data 634 for Channel 2 has been shifted from the original data 400 based on the temporal shift 610.

Next, at block 340, one or more of the augmented data for each channel for may be processed through one or more networks. The one or more networks may include but is not limited to one or more representation learning neural networks, one or projector networks (also referred to as “function g”), among others, or a combination thereof. In some examples, the one or more representation learning neural networks may include but is not limited to one or more of autoencoder neural networks, transformer neural networks, other latent variable models, among others, or any combination thereof. In some examples, the one or more projector networks may include but is not limited to a linear transformation network and/or a neural network configured to map the latent representations of one representation learning neural network to match the latent representations from another representation learning neural network.

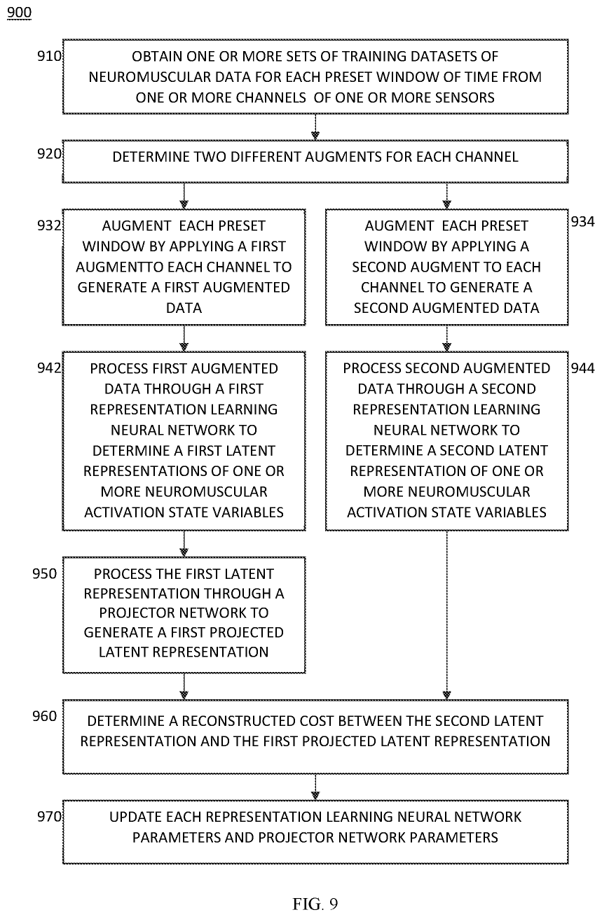

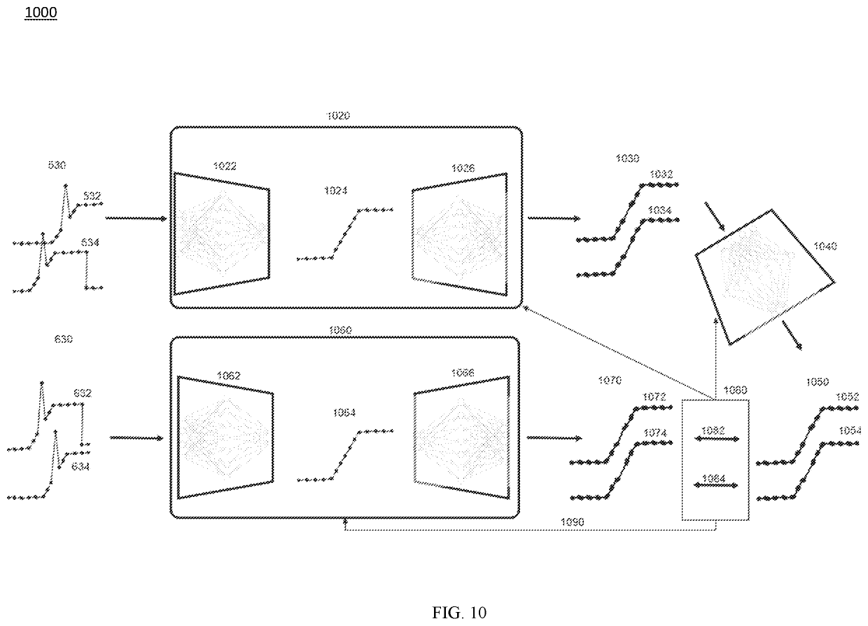

For example, one or more of the augmented data may be processed through a (trained) representation learning neural network to determine a latent representation of one or more neuromuscular activation state variables of the preset window of time for each channel. In some examples, at least the first augmented data may be processed through the representation learning neural network to generate a first latent representation of one or more neuromuscular activation state variables. In some examples, the second augmented data for each channel may also be processed through the same or different (trained) representation learning neural network to generate a second latent representation of one or more neuromuscular activation state variables. In some examples, the first latent representation for each channel may be processed through additional (trained) networks, such as a projector network (function g), for example, as shown in and described with respect to FIGS. 9 and 10.

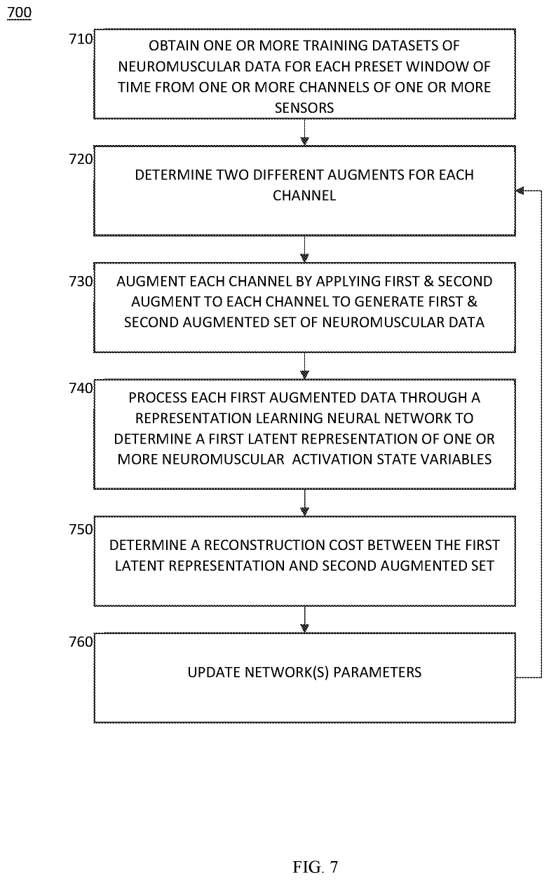

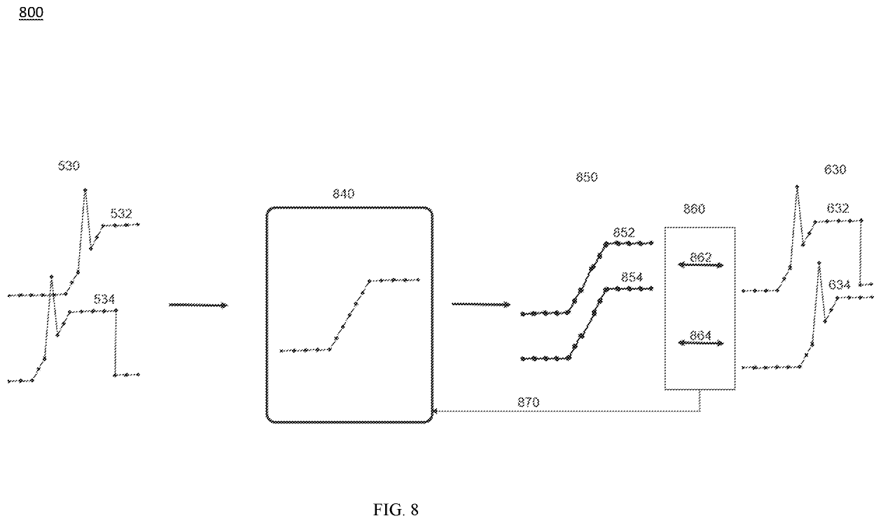

Next, at block 350, reconstruction cost (also referred to as “reconstruction loss”) may be determined for each preset window for each channel using at least an output of block 340. By way of example, the reconstruction cost can be determined using a log-likelihood cost function for a specific parameterized noise model (e.g., Poisson, Gaussian, Gamma, Binomial, etc.), mean-squared error (SE), among others, or any combination thereof. In some examples, for example, as shown in and described with respect to FIGS. 7 and 8, the reconstruction cost may be determined for each channel between the output of block 340 for the first augmented data (e.g., the first latent representation corresponding to the first augmented data) and the second augmented data from block 330. By way of another example, for example, as shown in and described with respect to FIGS. 9 and 10, the reconstruction cost can be determined between the outputs 340 (e.g., projected latent representation) for the first augmented data and (second latent representation) for the second augmented data for each channel.

Next, at block 360, based on the reconstruction cost, one or more parameters of the network(s) can be updated. For example, for each representation learning neural network, the one or more parameters may include but is not limited to weights of the linear transformation network parameters, neural network parameters, among others, or any combination thereof. For example, for the projector network, the one or more parameters may include but is not limited to weights of the linear transformation network parameters, neural network parameters, among others, or any combination thereof.

The training of the network(s), 320-360, may be repeated until the reconstruction cost determined at block 360 meets stopping criteria (e.g., the reconstruction costs have stopped changing after a set number of iterations).

The method 300 may also be used to train trained representation learning neural network(s) for a specific user. By way of example, the representation learning neural network may be initially trained using neuromuscular data from one or more users using the method 300. After which, neuromuscular data of the specific user may be used to further train the trained representation learning neural network(s) to that user using the method 300.

It will be understood that the steps 310-360 may be repeated for each window of data received. It will also be understood that steps 310-330 can be performed for each preset window can be performed in parallel.

It will also be understood that the data augmentation techniques disclosed herein to train on one or more networks (e.g., networks 112), for example, as discussed herein with respect to FIGS. 3 and 7-10 is not limited to being used with reconstruction cost (function) and can be used with different cost functions, such as those cost functions associated with contrastive learning, instance discrimination, and/or other self-supervised learning training paradigms.

FIG. 7 shows a flow chart 700 illustrating an example of a method for training one representation learning neural network using augmentation according to some embodiments. By way of example, the flow chart 700 can pertain to a generative model using reconstruction cost. FIG. 8 shows an illustrative example 700 of applying the method 700 to the augmented data for each channel.

In some examples, at block 710, like block 310, neuromuscular data for one or more preset windows of time (also referred to as “time window”) from one or more channels of one or more sensors (e.g., multi-channel and/or single-channel EMG sensor(s)) may be obtained for a subject.

Next, at block 720, like block 320, for each channel, one or more augments may be randomly generated for each channel. In some examples, two different augments may be randomly generated for each channel.

Next, at block 730, like block 330, the data for each preset window of each channel may be augmented by applying each augment to that data. In some examples, the neuromuscular data for each preset window of each channel may be augmented by applying a first augment to generate a first augmented data (x->x′) and by applying second, different augment to generate a second augmented data (x->x″) for each channel.

Next, at block 740, like block 340, one of the augmented data for each channel may be processed through one or more networks. In this example, the first augmented data may be processed through a (trained) representation learning neural network to determine a first latent representation (x′->y) of one or more neuromuscular activation state variables for the preset window of time of each channel.

Next, at block 750, reconstruction cost may be determined for each channel for each preset window using at least an output of block 740. By way of example, the reconstruction cost can be determined using a log-likelihood cost function for a specific parameterized noise model (e.g., Poisson, Gaussian, Gamma, Binomial, etc.), mean-squared error (SE), among others, or any combination thereof. In this example, the reconstruction cost may be determined between the second augmented data and the first latent representation corresponding to the first augmented data for each channel (y<->x″).

Next, at block 760, based on the reconstruction cost, one or more parameters of the network(s) can be updated. For example, for each representation learning neural network, the weights of the linear transformation network parameters and/or neural network parameters can be updated.

The training of the representation learning neural network, 720-760, may be repeated until the reconstruction cost determined at block 760 meets stopping criteria (e.g., the reconstruction costs has stopped changing after a set number of iterations).

FIG. 8 shows an example 800 of applying the method 700 to the augmented data 500 and 600 (e.g., data 400 augmented using randomly generated temporal shifts). As shown in FIG. 8, the first augmented data 530 may be processed through a representation learning neural network 840 to generate a first latent representation 850. Next, the total reconstruction cost 860 can be determined between second augmented data 630 and the first latent representation 850 of each channel. In this example, for Channel 1, the reconstruction cost 862 can be determined between the second augmented data 632 and the first latent representation 852 corresponding to the first augmented data 532; and for Channel 2, the reconstruction cost 864 can be determined between the second augmented data 634 and the first latent representation 854 corresponding to the first augmented data 534. Based on the total reconstruction cost 860 (e.g. 862 and 864), the parameters for the representation learning neural network 840 can be updated (870).

FIG. 9 shows a flow chart 900 illustrating an example of a method for training more than one network using temporal shift according to some embodiments. By way of example, the flow chart relates to a method for a self-supervised learning model. FIG. 10 shows an illustrative example 1000 of applying the method 900 to the augmented data for each channel.

In some examples, at block 910, like block 310, neuromuscular data for one or more preset windows of time from one or more channels of one or more sensors (e.g., single-channel and/or multi-channel EMG sensor(s)) may be obtained for a subject.

Next, at block 920, like block 320, for each channel, one or more augments may be randomly generated for each channel. In some examples, two augments may be randomly generated for each channel.

Next, at blocks 932 and 934, like block 330, the data for each preset window of each channel may be augmented by applying each augment to that data. In some examples, for each preset window of each channel, at block 932, the neuromuscular data may be augmented by applying a first augment to generate a first augmented data (x->x′) and at block 934, the neuromuscular data may also be augmented by applying second, different augment to generate a second augmented data (x->x″).

Next, at blocks 942 and 944, like block 340, one or more augmented data for each channel may be processed through one or more networks. In this example, each augmented data for each channel may be processed through a (trained) representation learning neural network to determine a latent representation of one or more neuromuscular activation state variables corresponding to each augmented data of each channel. For example, at block 942, the first augmented data may be processed through a first representation learning neural network to generate a first latent representation (x′->y′) and, at block 944, the second augmented data may be processed through a second (separate) representation learning neural network to generate a second latent representation (x″->y″).

Next, at block 950, one of those latent representations (e.g., a first latent representation (y′)) may be further processed by another network. For example, at block 950, the first latent representation may be processed by a projector network (e.g., function g) to generate a first projected latent representation (g(y′)). For example, please see Jean-Bastien Grill et al., 2020.

Next, at block 960, reconstruction cost may be determined using the outputs of blocks 944 and 950, for each preset window for each channel. For example, at block 960, the reconstruction cost may be determined between the second latent representation (output of block 944) and the first projected latent representation (output of block 960). By way of example, the reconstruction cost can be determined using a log-likelihood cost function for a specific parameterized noise model (e.g., Poisson, Gaussian, Gamma, Binomial, etc.), mean-squared error (SE), among others, or any combination thereof. In this example, the reconstruction cost may be determined between the second latent representation (y″) and the first projected latent representation (g(y′)).

Next, at block 970, based on the reconstruction cost, one or more parameters of each network can be updated. For example, the weights of the linear transformation network parameters and/or neural network parameters can be updated for each network, e.g., the first and second representation learning neural networks and the projector network.

Like the methods 300 and 700, the training of the networks, 920-970, may be repeated until the reconstruction cost determined at block 970 meets stopping criteria (e.g., the reconstruction costs has stopped changing after a set number of iterations).

FIG. 10 shows an example 1000 of applying the method 900 to the augmented data 500 and 600 (e.g., data 400 augmented using randomly generated temporal shifts). As shown in FIG. 10, the first augmented data 530 may be processed through a first representation learning neural network 1020 to generate a first latent representation 1030, and the second augmented data 630 may be process through a second representation learning neural network 1060 to generate a second latent representation 1070. In this example, each of the first representation learning neural network 1020 and the second representation learning neural network 1040 may be an autoencoder network. For example, the first representation learning neural network 1020 may include an encoder 1022, a latent variables (LV) model 1024, and a decoder 1026; and the second representation learning neural network 1060 may include an encoder 1062, a latent variables (LV) model 1064, and a decoder 1066. It will be understood that the first and/or second representation learning neural networks 1020 and 1060 may be separate representation learning neural networks.

Next, the first latent representation for each channel may further processed by a projector network (function (g)) 1040 to generate a first projected latent representation 1050. The total reconstruction cost 1080 can then be determined between the second latent representation 1070 and the first projected latent representation 1050 of each channel. In this example, for Channel 1, the reconstruction cost 1082 can be determined between the second latent representation 1072 and the first projected latent representation 1052; and for Channel 2, the reconstruction cost 1084 can be determined between the second latent representation 1074 and the first projected latent representation 1054. In some examples, based on the total reconstruction cost 1080, the parameters for each representation learning neural network 1020, 1060, respectively, can be updated (e.g., 1070 and 1090, respectively). The projector network 1040 can also be updated based on the total reconstruction cost 1080 (e.g., 1082 and 1084).

In some examples, as an alternative to using the total reconstruction cost 1080 to update the neural network parameters for the second representation learning neural network 1060, the second representation learning neural network 1060 parameters can also be updated based on a moving average of the values of the parameters of the first representation learning neural network 1020 over a preset number of training steps.

### EXAMPLES

As described above, the data augmentation as applied to training representation learning neural networks as described above can improve inference of muscle activation when monitored using neuromuscular data (e.g., EMG). A study has been conducted using techniques disclosed herein to train a representation learning neural network to model the spatial and temporal regularities that underlie multi-muscle activation, that results in better prediction of behavior (e.g., force, kinematics) and brain activity (i.e., motor cortical activity).

### Methods

### Section 1: Adapting AutoLFADS for EMG

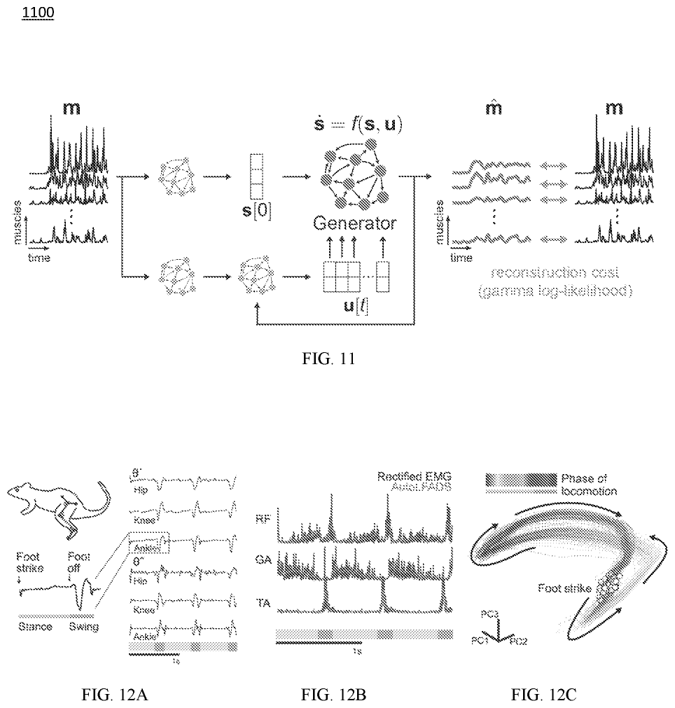

A model schematic outlining the adapted AutoLFADS approach is shown in FIG. 11. m[t] is the time-varying vector of rectified multi-muscle EMG. The goal of the study is to find m[t], an estimate of muscle activation from m[t] that preserves the neural command signal embedded in the recorded EMG activity. Common approaches to estimating m[t] typically involve applying a filter to each EMG channel independently, ranging from a simple linear smoothing filter to a nonlinear, adaptive Bayesian filter. While these approaches effectively reduce high-frequency noise, they have two major limitations. First, they require supervision to choose hyperparameters that affect the output of the filters. For example, using a smoothing (low-pass) filter requires a choice of a cutoff frequency and order, which sets an arbitrary fixed limit on the EMG frequency spectrum that is considered “signal”. Setting a lower cutoff yields smoother representations with less high-frequency content; however, this may also come at the expense of eliminating high-frequency activation patterns that may be behaviorally-relevant. Second, by filtering muscles independently, standard smoothing approaches fail to take advantage of potential information in the shared structure across muscles. Provided that muscles are not activated independently (Tresch and Jarc, 2009), the estimate of a given muscle's activation can be improved by having access to recordings from other muscles.

The study approach overcomes the limitations of standard filtering by leveraging the shared structure and potential nonlinear dynamics describing the time-varying patterns underlying m[t]. This approach was implemented by adapting latent factor analysis via dynamical systems (LFADS), a deep learning tool previously developed to study neuronal population dynamics (FIG. 11) (Pandarinath et al., 2018). Briefly, LFADS comprises a series of RNNs that model observed data as the noisy output of an underlying nonlinear dynamical system. The internal state of one of these RNNs, termed the Generator, explicitly models the evolution of s[t] i.e., the state of the underlying dynamical system. The Generator is trained to model f i.e., the state update function, by tuning the parameters of its recurrent connections to model the spatial and temporal regularities governing the coordinated time-varying changes of the observed data. The Generator does not have access to the observed data so it has no knowledge of the identity of recorded muscles or the observed behavior. Instead, the Generator is only provided with an initial state s[0], and a low-dimensional, time-varying input u[t] to model the progression of s[t] as a discrete-time dynamical system. The model then generates \hat{m}[t], where m[t] is taken as a sample of \hat{m}[t] through a gamma observation model (details are provided in AutoLFADS architecture). The model has a variety of hyperparameters that control training and prevent overfitting. These hyperparameters are automatically tuned using the AutoLFADS large-scale optimization framework previously developed (Keshtkaran et al., 2021; Keshtkaran and Pandarinath, 2019).

While the core LFADS model remains the same in its application for EMG activity as for cortical activity, there was no prior guarantee that adapting the observation model alone would provide us with good estimates of muscle activation from EMG. A key distinction to previous applications of LFADS is the application of data augmentation during model training. In the initial modeling attempts, it was found that models could be overfit to high-magnitude events that occurred simultaneously across multiple channels. To mitigate this issue, a data augmentation strategy (“temporal shift”) was implemented during model training to prevent the network from drawing a direct correspondence between specific values of the rectified EMG used both as the input to the model and at the output to compute the reconstruction cost (details provided in AutoLFADS training).

### AutoLFADS Network Architecture

The AutoLFADS network architecture, as adapted for EMG, is detailed in Supplementary FIG. 1, Lahiru N. Wimalasena et al. 2022. Estimating muscle activation from EMG using deep learning-based dynamical systems models. J. Neural Eng. 19 (3) 036013, which is hereby incorporated by reference in its entirety. The model operates on segments of multi-channel rectified EMG data (m[t]). The output of the model \hat{m}[t] is an estimate of muscle activation from m[t] in which the observed activity on each channel is modeled as a sample from an underlying time-varying gamma distribution. For channel j, \hat{m}ⱼ[t] is the time-varying mean of the gamma distribution inferred by the model (i.e., AutoLFADS output). The two parameters for each gamma distribution, denoted a (concentration) and β (rate), are taken as separate linear transformations of the underlying dynamic state s[t] followed by an exponential nonlinearity. Because rectified EMG follows a skewed distribution (i.e., values are strictly positive), data were log transformed before being input to the model, resulting in data distributions that were more symmetric.

To generate s[t] for a given window of data m[t], a series of RNNs, implemented using Gated Recurrent Unit (GRU) cells, were connected together to explicitly model a dynamical system. At the input to the network, two RNNs (the Initial Condition & Controller Input Encoders) operate on the log-transformed m[t] to generate an estimate of the initial state s[0] and to encode information for a separate RNN, the Controller. The role of the controller is to infer a set of time-varying inputs into the final RNN, the Generator, u[t]. This Generator RNN represents the learned dynamical system, which models f to evolve s[t] at each timepoint. From s[0], the dynamical system is run forward in time with the addition of the u[t] at each timestep to evolve s[t].

In all experiments performed in this paper, the Generator, Controller, and Controller Input Encoder RNNs each had 64 units. The Initial Condition Encoder RNN had a larger dimensionality of 128 units to increase the capacity of the network to handle the complex mapping from the unfiltered rectified EMG onto the parameters of the initial condition distribution. The Generator RNN was provided with a 30-dimensional initial condition (i.e., s[0]) and a three-dimensional input (i.e., u[t]) from the Controller. A linear readout matrix restricted the output of the Generator RNN to describe the patterns of observed muscle activity using only 10 dimensions. Variance of the initial condition prior distribution was 0.1. The minimum variance of the initial condition posterior distribution was 1e−4.

### AutoLFADS Training

Training AutoLFADS models on rectified EMG data largely followed previous applications of LFADS (Keshtkaran et al., 2021; Keshtkaran and Pandarinath, 2019; Pandarinath et al., 2018; Sussillo et al., 2016), with key distinctions being the gamma emissions process chosen for EMG data, as described above, and a novel data augmentation strategy implemented during training (see below). Beyond this choice, model optimization followed previous descriptions; briefly, the model's objective function is defined as the log likelihood of the observed EMG data (m[t]) given the time-varying gamma distributions output by the model for each channel (\hat{m}[t]), marginalized over all latent variables. This is optimized in a variational autoencoder by maximizing a lower bound on the marginal data log-likelihood (Kingma and Welling, 2014). Network weights were randomly initialized. During training, network parameters are optimized using stochastic gradient descent and backpropagation through time. The loss prior to computing the gradients (scale: 1e4) was scaled to prevent numerical precision issues. The Adam optimizer (epsilon: 1e−8, beta1: 0.9, beta2: 0.99) was used to control weight updates, and implemented gradient clipping to prevent potential exploding gradient issues (max. norm=200). To prevent any potentially problematic large values in RNN state variables and achieve more stable training, the range of the values in GRU hidden state was also limited by clipping values greater than 5 and lower than −5.

Hyperparameter optimization. Finding a high-performing model required finding a suitable set of model hyperparameters (HPs). To do so, an automated model training and hyperparameter framework that was developed (Keshtkaran and Pandarinath, 2019), which is an implementation of the population based training (PBT) approach (Jaderberg et al., 2017), was used. Briefly, PBT is a large-scale optimization method that trains many models with different HPs in parallel. PBT trains models for a generation—i.e., a user-settable number of training steps-then performs a selection process to choose higher performing models and eliminate poor performers. Selected high performing models are also “mutated” (i.e., their HPs are perturbed) to expand the HP search space. After many generations, the PBT process converges upon a high performing model with optimized HPs.

For both datasets, we used PBT to simultaneously train 20 LFADS models for 30 epochs per generation (where an “epoch” is a single training pass through the entire dataset). We distributed training across 5 GPUs (4 models were trained on each GPU simultaneously). The magnitude of the KL and L2 penalties was linearly ramped for the first 50 epochs of training during the first generation of PBT. Each training epoch consisted of ˜10 training steps. Each training step was computed on 400-sample batches from the training data. PBT stopped when there was no improvement in performance after 10 generations. Runs typically took ˜30-40 generations to complete. After completion, the model with the lowest validation cost (i.e., best performance) was returned and used as the AutoLFADS model for each dataset. AutoLFADS output was inferred 200 times for each model using different samples from initial condition and controller output posterior distributions. These estimates were then averaged, resulting in the final model output used for analysis.

PBT was allowed to optimize the model's learning rate and five regularization HPs: L2 penalties on the Generator and Controller RNNs, scaling factors for the KL penalties applied to the initial conditions and time-varying inputs to the Generator RNN, and two dropout probabilities (see PBT Search Space in Supplementary Methods of Lahiru N. Wimalasena et al. 2022 for specific ranges).

Data augmentation. A key distinction of the AutoLFADS approach for EMG data with respect to previous applications of LFADS was the development and use of data augmentation strategies during training. In our initial modeling, it was found that models could be overfit to high-magnitude events that occurred simultaneously across multiple channels. To prevent this overfitting, a novel data augmentation strategy (“temporal shift”) was implemented. During model training, each channel's data we randomly shifted, i.e., small, random temporal offsets (selected separately for each channel) was introduced when inputting data to the model. The model input and the data used to compute reconstruction cost were shifted differently to prevent the network from overfitting by drawing a direct correspondence between specific values of the input and output (similar to the problem described in Keshtkaran and Pandarinath, 2019). During temporal shifting, each channel's data was allowed to shift in time by a maximum of 6 bins. Shift values were drawn from a normal distribution (std. dev.=3 bins) that was truncated such that any value greater than 6 was discarded and re-drawn.

### Section 2: Data

### Rat Locomotion Data

EMG recordings. Chronic bipolar EMG electrodes were implanted into the belly of 12 hindlimb muscles during sterile surgery as described in (Alessandro et al., 2018). Recording started at least two weeks after surgery to allow the animal to recover. Differential EMG signals were amplified (1000×), band-pass filtered (30-1000 Hz) and notch filtered (60 Hz), and then digitized (5000 Hz).

Kinematics. A 3d motion tracking system (Vicon) was used to record movement kinematics (Alessandro et al., 2020, 2018). Briefly, retroreflective markers were placed on bony landmarks (pelvis, knee, heel, and toe) and tracked during locomotion at 200 Hz. The obtained position traces were low-pass filtered (Butterworth 4th order, cut-off 40 Hz). The 3-D knee position was estimated by triangulation using the lengths of the tibia and the femur in order to minimize errors due to differential movements of the skin relative to the bones (Bauman and Chang, 2010; Filipe et al., 2006). The 3d positions of the markers were projected into the sagittal plane of the treadmill, obtaining 2d kinematics data. Joint angles were computed from the positions of the top of the pelvis (rostral marker), the hip (center of the pelvis), the knee, the heel and the toe as follows. Hip: angle between the pelvis (line between pelvis-top and hip) and the femur (line between hip and knee). Knee: angle between the femur and the tibia (line between the knee and the heel). Ankle: angle between the tibia the foot (line between the heel and the toe). All joint angles are equal to zero for maximal flexion and increase with extension of the joints.

Joint angular velocities and accelerations were computed from joint angular position using a first-order and second-order Savitzky-Golay (SG) differentiation filter (MATLAB's sgolay), respectively. SG FIR filter was designed with a 5th order polynomial and frame length of 27.

### Monkey Isometric Data

M1/EMG Recordings. Data from one 10 kg male macaca mulatta monkey (age 8 years when the experiments started) while he performed an isometric wrist task was recorded. All surgical and behavioral procedures were approved by the Animal Care and Use Committee at Northwestern University. The monkey was implanted with a 96-channel microelectrode silicon array (Utah electrode arrays, Blackrock Microsystems, Salt Lake City, UT) in the hand area of M1, which we identified intraoperatively through microstimulation of the cortical surface and identification of surface landmarks. The monkey was also implanted with intramuscular EMG electrodes in a variety of wrist and hand muscles. The data was reported from the following muscles: flexor carpi radialis, flexor carpi ulnaris, flexor digitorum profundus (2 channels), flexor digitorum superficialis (2 channels), extensor digitorum communis, extensor carpi ulnaris, extensor carpi radialis, palmaris longus, pronator teres, abductor pollicis brevis, flexor pollicis brevis, and brachioradialis. Surgeries were performed under isoflurane gas anesthesia (1-2%) except during cortical stimulation, for which the monkeys were transitioned to reduced isoflurane (0.25%) in combination with remifentanil (0.4 μg kg−1 min−1 continuous infusion). Monkeys were administered antibiotics, anti-inflammatoires, and buprenorphine for several days after surgery.

Experimental Task. The monkey was trained at a 2D isometric wrist task for which the relationship between muscle activity and force is relatively simple and well characterized. The left arm was positioned in a splint so as to immobilize the forearm in an orientation midway between supination and pronation (with the thumb upwards). A small box was placed around the monkey's open left hand, incorporating a 6 DOF load cell (20E12, 100 N, JR3 Inc., CA) aligned with the wrist joint. The box was padded to comfortably constrain the monkey's hand and minimize its movement within the box. The monkey controlled the position of a cursor displayed on a monitor by the force they exerted on the box. Flexion/extension force moved the cursor right and left, respectively, while forces along the radial/ulnar deviation axis moved the cursor up and down. Prior to placing the monkey's hand in the box, the force was nulled in order to place the cursor in the center target. Being supported at the wrist, the weight of the monkey's hand alone did not significantly move the cursor when the monkey was at rest. Targets were displayed either at the center of the screen (zero force), or equally spaced along a ring around the center target. All targets were squares with 4 cm sides.

### Data Preprocessing

Raw EMG data was first passed through a powerline interference removal filtering algorithm (Keshtkaran and Yang, 2014) that we specified to remove power at 60 Hz and 2 higher frequency harmonics (120 Hz, 180 Hz). Additional notch filters centered at 60 Hz and 94 Hz (isometric data) and 60 Hz, 120 Hz, 240 Hz, 300 Hz, and 420 Hz (locomotion data) were added to remove noise peaks identified from observing the power spectral density estimates after the previous filtering step. The EMG data was then filtered with a 4th-order Butterworth high pass filter with cutoff frequency at 65 Hz. The EMG and down-sampled from the original sampling rate (rat: 5 KHz, monkey: 2 KHz) was finally full-wave rectified to 500 Hz using MATLAB's resample function which applies a poly-phase anti-aliasing filter prior to down-sampling the data.

Next, infrequent electrical recording artifacts that caused large spikes in activity which had a particular effect on AutoLFADS modeling were removed: during the modeling process (see Training), large anomalous events incurred a large reconstruction cost that disrupted the optimization process and prevented the model from capturing underlying signal. To remove artifacts, each channel's activity was clipped such that any activation above a threshold value was set to the threshold. For the isometric data, the same 99.99th percentile threshold was applied across all channels. For the locomotion data, we used a 99.9th percentile threshold for all channels except for one muscle (semimembranosus) which required a 99th percentile threshold to eliminate multiple high-magnitude artifacts only observed in the recordings for this channel. After clipping, each channel was normalized by the 95th percentile value to ensure that they had comparable variances.

For the monkey data, some artifacts remained even after clipping. To more thoroughly reject artifacts, windows of data were selected for successful trials (1.5 s prior to 2.5 s after force onset) and visually screened all muscles. Trials in which muscle activity contained high magnitude events (brief spikes 2-5× greater than typical range) that were not seen consistently across trials were excluded for subsequent analyses. After the rejection process, 271 of 296 successful trials remained.

AutoLFADS was used to model the EMG data without regard to behavioral structure (i.e., task or trial information). For the rodent locomotion data, the continuously recorded data (˜2 min per session) from three speed conditions (10, 15, 20 m/min) was binned at 2 ms and divided into windows of length 200 ms, with 50 ms of overlap between the segments. The start indices of each trial are stored so that after training, model output can be reassembled. After performing inference on each segment separately, the segments were merged at overlapping regions using a linear combination of the estimates from the end of the earlier segment with those from the beginning of the later segment. The merging technique weighted the ends of the segments as w=1−x and the beginning of segments as 1−w, with x linearly increasing from 0 to 1 across the overlapping points. After weights were applied, the overlapping segments were summed to reconstruct the modeled data. Non-overlapping portions of the windows were concatenated to the array holding the reconstructed continuous output of the model, without any weighting. Due to the additional artifact rejection process for the monkey isometric data (mentioned above), we were unable to model the continuously recorded data (i.e., we could only model segments corresponding to trials that passed the artifact rejection process). These trial segments were processed using the same chopping process we administered to the rodent locomotion data.

### Bayesian Filtering

Model. Bayesian filtering is an adaptive filtering method, that, depending on the model specifications, can be linear (Kalman filter) or nonlinear, and has previously been applied to surface EMG signals (Dyson et al., 2017; Hofmann et al., 2016; Sanger, 2007). A Bayesian filter consists of two components: (1) the temporal evolution model governing the time-varying statistics of the underlying latent variable and (2) the observation model that relates the latent variable to the observed rectified EMG. For this application, the temporal evolution model was implemented using a differential Chapman Kolmogorov equation reflecting diffusion and jump processes (Hofmann et al., 2016). For the observation model, both a Gaussian distribution and Laplace distribution, which have previously been applied to surface EMG (Nazarpour et al., 2013) was tested. Empirically, it was found that the Gaussian distribution produced slightly better decoding predictions (for both rat and monkey) than the Laplace distribution, so it was used for all comparisons. The study's implementation of the Bayesian filter had four hyperparameters: (1) sigmamax, the upper interval bound for the latent variable, (2) Nbins, the number of bins used to define the histogram modeling the latent variable distribution, (3) alpha, which controls the diffusion process underlying the time-varying progression of the latent variable and (4) beta, which controls the probability for larger jumps of the latent variable. Sigmamax was empirically derived to be approximately an order of magnitude larger than the standard deviation measured from the aggregated continuous data. This value was set to 2 for both datasets after quantile normalizing the EMG data prior to modeling. Nbins was set to 100 to ensure the changes in the magnitude of the latent variable over time could be modeled with high enough resolution. The other two hyperparameters, alpha and beta, had a significant effect on the quality of modeling, therefore we performed a large-scale grid search to optimize the parameters for each dataset (described below). Each EMG channel is estimated independently using the same set of hyperparameters.

Hyperparameter optimization. Two hyperparameters (alpha and beta) can be tuned to affect the output of the Bayesian filter. To ensure that the choice to use previously published hyperparameters (Sanger, 2007) was not resulting in sub-optimal estimates of muscle activation, we performed grid searches for each dataset to find an optimal set of hyperparameters to filter the EMG data. We tested 4 values of alpha (ranging from 1e−1 to 1e−4) and 11 values of beta (ranging from 1e0 to 1e−10), resulting in 44 hyperparameter combinations. To assess optimal hyperparameters for each dataset, linear decoders from the Bayesian filtered EMG were fit to predict joint angular acceleration (rat locomotion) or dF/dt (monkey isometric) following the same procedures used for decoding analyses (see Predicting joint angular acceleration or Predicting dF/dt for details). The hyperparameter combination that resulted in the highest average decoding performance across prediction variables was chosen for comparison to AutoLFADS. These were alpha=1e−2 and beta=1e−4 (rat locomotion) and alpha=1e−2 and beta=1e−2 (monkey isometric).

### Section 3: Analyses

Metric. To quantify decoding performance, Variance Accounted For (VAF) was used. VAF=1−Σ(ŷ−y)²/Σ(y−y̅). ŷ is the prediction, y is the true signal, and y̅ is the mean. This is often referred to as the coefficient of determination, or R².

Predicting joint angular acceleration (rat locomotion). Optimal linear estimation (OLE) was used to perform single timepoint decoding to map the muscle activation estimates of 12 muscles onto the 3 joint angular acceleration kinematic parameters (hip, knee, and ankle) at a single speed (20 cm/s). A 10-fold cross validation (CV) was performed to fit decoders (90% of the data) and evaluate performance (held-out 10% of data). Mean and standard error of the mean (SEM) for decoding performance was reported as the averaged cross-validated VAF across the 10 folds. During fitting of the decoders, the predictor (i.e., muscle activation estimate) and response (i.e., joint angular kin) variables was normalized to have zero-mean and unit standard deviation. The weights of the decoder was also penalized using an L2 ridge regularization penalty of 1. Significance was assessed using paired t-tests between the cross-validated VAF values from the AutoLFADS predictions and the predictions from the best performing low pass filter (10 CV folds). A sweep ranging from 0 ms to 100 ms was performed to find the optimal lag for each predictor with respect to each response variable. The lag of each decoder was separately optimized to account for potential differences in the delay between changes in the muscle activation estimates and changes in the joint angular kinematics, which could be different for each kinematic parameter. Optimal lag was determined based on the highest mean CV VAF value.

Estimating frequency responses. The frequency responses was estimated to understand how AutoLFADS differs from low pass (Butterworth) filtering, which applies the same temporally invariant frequency response to all channels. The goal was to see whether AutoLFADS optimizes its filtering properties differently for individual muscles around characteristic timepoints of behavior.

Two characteristic time points were chosen within the gait cycle of the rat locomotion dataset: (1) during the swing phase (at 25% of the time between foot off and foot strike) and (2) during the stance phase (50% between foot off and foot strike). We selected windows of 175 samples (350 ms) around both of these time points. The number of samples was optimized to be large enough to have a fine resolution in frequency space but small enough to include samples mainly from one of the locomotion phases (avg. stance: ˜520 ms; avg. swing: ˜155 ms). To reduce the effects of frequency resolution limits imposed by estimating PSDs on short sequences, we used a multi-taper method that matches the length of the windows used for the PSD estimates to the length of the isolated segment. This maximized the number of points of the isolated segments we could use without upsampling. We z-scored the signals within each phase and muscle to facilitate comparisons. Within each window, we computed the Thomson's multitaper power spectral density (PSD) estimate using a MATLAB (Mathworks Inc, MA) built-in function pmtm. The time-bandwidth product was set to NW=3 to retain precision in frequency space and the number of samples for the DFT to equal the window length and otherwise retained default parameters. With a window of 175 samples, the DFT has a frequency resolution of 2.85 Hz.

The PSD was computed for the rectified EMG (PSD_{raw}), the output of AutoLFADS (PSD_{AutoLFADS}) and the output of a 20 Hz Butterworth low pass filter (PSD_{lƒ20}) within each window. The linear frequency responses were then computed as a ratio of the filtered and raw spectra. Finally, we computed the mean and SEM across each EMG and gait cycle.

Predicting dF dt (monkey isometric). Force onset was determined within a window 250 ms prior and 750 ms after the target appearance in successful trials by finding the threshold crossing point 15% of the max dF/dt within that window. OLE was used to perform single timepoint decoding to map the EMG activity of 14 muscle channels onto the X and Y components of dF/dt. The same CV scheme used in the rat locomotion joint angular decoding analysis was performed to evaluate decoding performance of the different EMG representations. During fitting of the decoders, the EMG predictors were normalized to have zero-mean and unit standard deviation. The weights of the decoder were penalized using an L2 ridge regularization penalty of 1.

Predicting M1 activity. OLE was used as described above to perform single timepoint linear decoding to map the EMG activity of 14 muscle channels onto the 96 channels of Gaussian smoothed motor cortical threshold crossings (standard deviation of Gaussian kernel: 35 ms). A sweep was performed for the optimal lag (ranging from 0 to 150 ms) for each M1 channel. To assess significance, a Wilcoxon signed-rank test was performed using MATLAB command signrank to perform a one-sided nonparametric paired difference test comparing the mean CV VAF values for predictions of all 96 channels.

Oscillation analyses. To visualize the consistency of the oscillations observed in AutoLFADS output from the monkey isometric task, the lead or lag for single trials was varied in order to minimize the residual between the single trial activity and the trial-average for a given condition (Williams et al., 2020). The algorithm was provided with a window of AutoLFADS output around the time of force onset (80 ms prior, 240 ms after), and constrained the algorithm to limit the maximum shift to be 35 ms, approximately half the width of an oscillation, and for the trial-averaged templates for each condition to be non-negative. For all applications of the optimal shift algorithm, a smoothness penalty of 1.0 was applied on the L2 norm of second temporal derivatives of the warping templates.

For coherence analyses, the single-trial data around the alignment provided by the optimal shifting algorithm and isolated a window of EMG activity and dF/dt (300 ms prior, 600 ms after) was aligned. For each condition, the MATLAB function mscohere was used to compute coherence between the single trial activity of a given muscle's activity and dF/dt X or Y. The Hanning window size and the number of FFT points (120 timepoints) were matched and 100 timepoints of overlap between windows were used.

### Results

Applying AutoLFADS to EMG from Locomotion

The adapted AutoLFADS approach was first tested using previously recorded data from the right hindlimb of a rat walking at constant speed on a treadmill (Alessandro et al., 2018). Seven markers were tracked via high-speed video, which allowed the 3 joint angular kinematic parameters (hip, knee, and ankle) to be inferred, along with the time of foot contact with the ground (i.e., foot strike) and the time when the foot left the ground (i.e., foot off) for each stride (FIG. 12a). EMG signals from 12 hindlimb muscles were also recorded using intramuscular electrodes. Continuous hindlimb EMG recordings were segmented using behavioral event annotations, considering foot strike as the beginning of each stride. Aligning estimates to behavioral events allowed us to decompose individual strides.

AutoLFADS output substantially reduced high-frequency fluctuations while still tracking fast changes in the EMG (FIG. 12b), reflecting both slowly changing phases of the behavior (e.g., the stance phase) and rapid behavioral changes (e.g., the beginning of the swing phase. AutoLFADS models were trained in a completely unsupervised manner, which meant the models did not have information about the behavior, the identity of the recorded muscles, or any additional information outside of the rectified multi-muscle EMG activity. Visualizing muscle activity using state space representations helps to illustrate how AutoLFADS was capable of modeling dynamics. Dimensionality reduction was performed via principal components analysis to reduce the 12 AutoLFADS output channels to the top three components (FIG. 12c). AutoLFADS estimates from individual strides followed a stereotyped trajectory through state space: state estimates were tightly clustered at the time of foot strike, and evolved in a manner where activity at each phase of the gait cycle occupied a distinct region of state space.

### AutoLFADS Adapts Filtering to Preserve Behaviorally Relevant, High-Frequency Features

A key feature that differentiates AutoLFADS from previous approaches is its ability to adapt its filtering properties dynamically to phases of behavior having signal content with different timescales. It was demonstrated the adaptive nature of the AutoLFADS model by comparing its filtering properties during the swing and stance phases of locomotion. Swing phase contains higher-frequency transient features related to foot lift off and touch down, which contrast with the lower frequency changes in muscle activity during stance. We computed the power spectral density (PSD) for the AutoLFADS inferred muscle activations, rectified EMG smoothed with a 20 Hz low pass 4-pole Butterworth filter, and the unfiltered rectified EMG within 350 ms windows centered on the stance and swing phases of locomotion. These PSD estimates were then used to compute the frequency responses of the AutoLFADS model—i.e., approximating it as a linear filter—and of the 20 Hz low pass filter over many repeated strides within the two phases (see Methods).

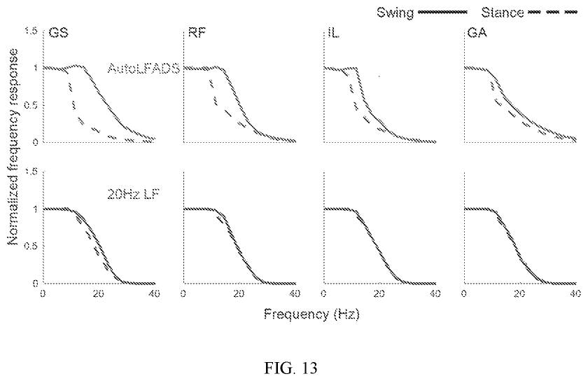

As expected, the 20 Hz low pass filter had a consistent frequency response during the swing and stance phases (FIG. 13). In contrast, the AutoLFADS output resulted in substantially different frequency response estimates that enhanced high frequencies during the rapid changes of the swing phase and attenuated them during stance. Less obviously, the frequency responses estimated for AutoLFADS differed across muscles, despite its reliance on a shared dynamical system. Some AutoLFADS frequency responses (e.g., GS and RF) changed substantially between stance and swing in the 10-20 Hz frequency band; however, other muscles (e.g., IL and GA) shifted only slightly.

AutoLFADS is More Predictive of Behavior than Other Filtering Approaches

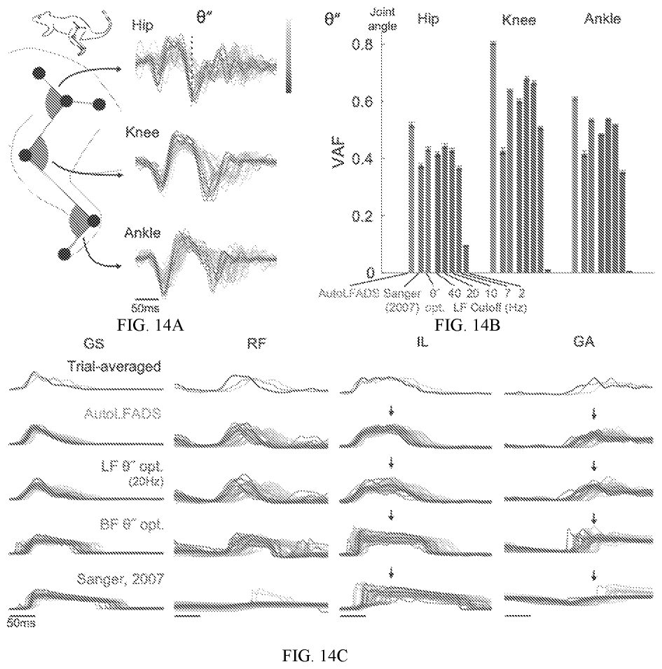

Training AutoLFADS models in an unsupervised manner (i.e., without any information about simultaneously recorded behavior, experiment cues, etc.) raises the question of whether the muscle activation estimates were informative about behavior. To assess this, muscle activations estimated by AutoLFADS were compared to those estimated using standard filtering approaches, by evaluating how well each estimate could decode simultaneously recorded behavioral variables (FIG. 14). A single-timestep optimal linear estimation (OLE) was performed on joint angular accelerations (FIG. 14a) that captured high-frequency changes in the joint angular kinematics of rats during locomotion.

AutoLFADS significantly outperformed all low-pass filter cutoffs (ranging from 2 to 40 Hz), as well as Bayesian filtering for prediction of angular acceleration at the hip, knee, and ankle joints, as quantified by the variance accounted for (VAF; FIG. 14b). AutoLFADS substantially outperformed the next-highest performing method for all joints (differences in VAF: hip: 0.10, knee: 0.13, ankle: 0.06, p<1e−05 for all comparisons, paired t-test). Visualizing the muscle activation estimates for individual strides underscored the differences between the approaches (FIG. 14c). Even Bayesian filters optimized to maximize prediction accuracy of joint angular accelerations did significantly worse than AutoLFADS. Qualitatively, it was found that the Bayesian filter overestimated the amount of time each muscle was active during the gait cycle. Even though the AutoLFADS model was trained in an unsupervised manner with no knowledge of the joint angular accelerations, the study approach outperformed other approaches that had manually tuned hyperparameters to yield the best possible decoding performance. Separately, it was found that AutoLFADS generalized well across behavioral conditions for example, by training on data from a single condition (locomotion speed, or treadmill incline) and testing on data from unseen conditions (detailed in Supplementary FIG. 2 of Lahiru N. Wimalasena et al. 2022 for specific ranges). Perhaps surprisingly, we also found that performance was robust to dataset size limitations (Supplementary FIG. 3 of Lahiru N. Wimalasena et al. 2022 for specific ranges).

Applying AutoLFADS to Forearm EMG from Isometric Wrist Tasks

To test the generalizability of our approach to other model systems, muscle groups, and behaviors, data from a monkey performing a 2-D isometric wrist task (FIG. 15a) (Gallego et al., 2020) was also analyzed. EMG activity was recorded from 12 forearm muscles (with multiple recordings in two muscles) during a task requiring the monkey to generate isometric forces at the wrist. The monkey's forearm was restrained and its hand placed securely in a box instrumented with a 6 DOF load cell. Brain activity was simultaneously recorded via a 96-channel microelectrode array in the hand area of contralateral M1. The monkey controlled the position of a cursor displayed on a monitor by modulating the force they exerted. Flexion and extension forces moved the cursor right and left, respectively, while forces along the radial and ulnar deviation axis moved the cursor up and down. The monkey controlled the on-screen cursor to acquire targets presented in one of eight locations in a center-out behavioral task paradigm. Each trial consisted of a step-like transition from a state of minimal muscle activity prior to target onset to one of eight phasic-tonic muscle activation patterns (i.e., one for each target) that enabled the monkey to hold the cursor at the target. The distinct, step-like muscle activation patterns across targets contrasted the cyclic behavior and highly repetitive activation patterns observed during rat locomotion, allowing us to evaluate AutoLFADS in two fundamentally different contexts.

An AutoLFADS model was trained on EMG activity from the monkey forearm during the wrist isometric task to estimate the muscle activation for each muscle (FIG. 15b). As was the case for the locomotion data, AutoLFADS substantially reduced high-frequency fluctuations while still tracking changes in the rectified EMG, reflecting both smooth phases of the behavior (e.g., the period prior to target onset in the wrist task) and rapid behavioral changes (e.g., the target acquisition period in the wrist task). Dimensionality reduction was again performed to reduce the 14 AutoLFADS outputs to the top three PCs. In the monkey data, the muscle activation estimates from AutoLFADS began consistently in a similar region of state space at the start of each trial, then separated to different regions at the target hold period. This demonstrates that the model was capable of inferring distinct patterns of muscle activity for different target directions, even without the cyclic, repeated task structure of the locomotion data (FIG. 15c).

### AutoLFADS Uncovers High-Frequency Oscillatory Structure Across Muscles During Isometric Force Production

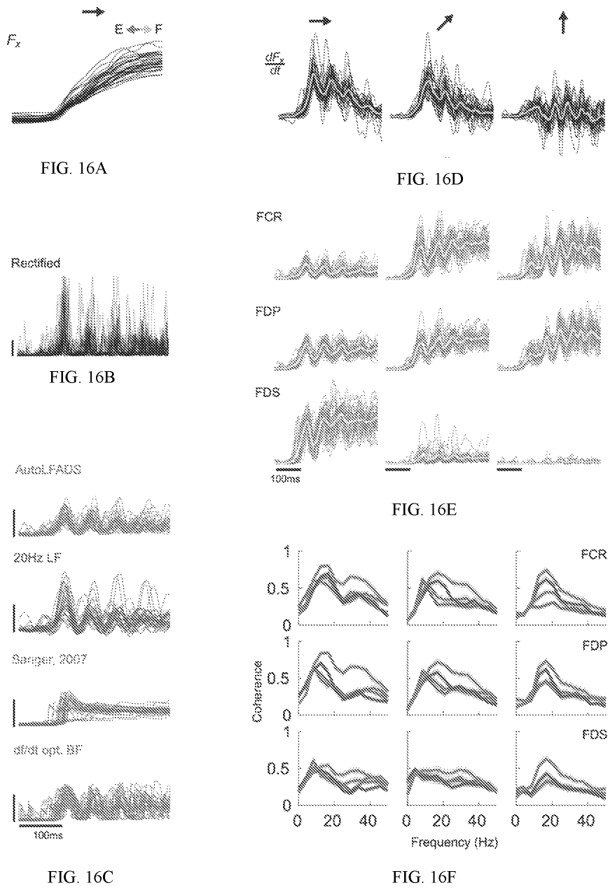

An interesting attribute of the isometric wrist task is that for certain target conditions that require significant flexion of the wrist, the x component of force contains high-frequency oscillations-made evident by visualizing the dF/dt (FIG. 16a). Corresponding oscillatory structure can be seen in the EMG of flexor muscles (FIG. 16b). It was found that muscle activation estimates of these high-frequency features differed across approaches (FIG. 16c) AutoLFADS output showed clear, consistent oscillations that lasted for 3-4 cycles, while other approaches such as Bayesian filtering (using the hyperparameters from Sanger, 2007) smoothed over these features. Oscillations in dFₓ/dt were visible for three of the eight target conditions (FIG. 16d) and corresponding oscillations were observed across the flexor muscles in the AutoLFADS output (FIG. 16e) in those trials.

To quantify this correspondence, we computed the coherence between the muscle activation estimates and dFₓ/dt separately for each condition for single trials. If the high-frequency features conserved by AutoLFADS accurately reflect underlying muscle activation, then they should have a closer correspondence with behavior (i.e., dFₓ/dt) than those features that remain after smoothing or Bayesian filtering. Coherence in the range of 10-50 Hz was significantly higher for AutoLFADS than for the other tested approaches, while all three muscle activation estimates had similar coherence with dF/dt below 10 Hz (FIG. 16f). These results further demonstrate the power of AutoLFADS to capture high-frequency features in the muscle activation estimates with higher fidelity than previous approaches.

AutoLFADS Preserves Information about Descending Cortical Commands

Simultaneous recordings of motor cortical and muscle activity gave us the ability to test how accurately AutoLFADS could capture the influence of descending cortical signals on muscle activation. We trained linear decoders to map the different muscle activation estimates onto each of the 96 channels of smoothed motor cortical activity. Note that while the causal flow of activity is from M1 to muscles, we predicted in the opposite direction (muscles to M1) so that we had a consistent target (M1 activity) to assess the informativeness of the different muscle activation estimates. For each channel of M1 activity, the observed threshold crossings were smooth using a Gaussian kernel with standard deviation of 35 ms. Predictions were performed in a window spanning 150 ms prior to 150 ms after force onset. For each neural channel, it was found the optimal single decoding lag between neural and muscle activity by sweeping in a range from 0 to 100 ms (i.e., predicted neural activity was allowed to precede the muscle activity predictors by up to 100 ms). AutoLFADS significantly improved the accuracy of M1 activity prediction when compared to 20 Hz low pass filtering (FIG. 17; mean percentage improvement: 12.4%, p=2.32e−16, one-sided Wilcoxon paired signed rank test). Similarly, AutoLFADS significantly improved M1 prediction accuracy when compared to Bayesian filtering using previously published hyperparameters (mean percentage improvement: 22.2%, p=4.58e−16, one-sided Wilcoxon paired signed rank test) or using hyperparameters optimized to predict behavior (mean percentage improvement: 16.9%, p=7.37e−16, one-sided Wilcoxon paired signed rank test). This demonstrates that AutoLFADS not only captures behaviorally-relevant features, but also preserves signals that reflect activity in upstream brain areas that are involved in generating muscle activation commands.

### Discussion

### Relation to Previous Work

Substantial effort has been invested over the past 50 years to develop methods to estimate the latent command signal to muscles—termed the neural drive—through EMG recordings (Farina et al., 2014, 2004). This is a challenging problem as typical recordings reflect the summed activity of many different motor units, which can lead to interference among the biphasic potentials, and signal cancellation (Negro et al., 2015). As muscle contraction increases, the linear relationship between the number of active motor units and the amplitude of the recorded EMG becomes distorted, making it non-trivial to design methods that can extract neural drive (Dideriksen and Farina, 2019).

Higher spatial resolution recordings that isolate individual motor units may allow neural drive to be inferred with greater precision. For example, high-density EMG arrays have enabled access to as many as 30 to 50 individual motor units from the surface recordings of a single muscle (De Luca et al., 2006; Del Vecchio et al., 2020; Farina et al., 2016). By isolating, then recombining the activity of individual motor units, one can better extract the common input signal, yielding estimates that are more closely linked to force output than is the typical EMG (Boonstra et al., 2016; De Luca and Erim, 1994; Farina and Negro, 2015). Here, we showed that AutoLFADS similarly enhanced correspondence to force output and other behavioral parameters (e.g., joint angular acceleration) relative to standard EMG processing techniques; how well this improvement approaches that of inferring and recombining multiple motor unit recordings is not clear and should be the goal of future work.

AutoLFADS leverages recent advances at the intersection of deep learning and neuroscience to create a powerful model that describes both the spatial and temporal regularities that underlie multi-muscle coordination. Previous work has attempted to estimate neural drive to muscles by exploiting either temporal regularities in EMGs using Bayesian filtering techniques applied to single muscles (Hofmann et al., 2016; Sanger, 2007) or spatial regularities in the coordination of activity across muscles (Alessandro et al., 2012; d'Avella et al., 2003; Hart and Giszter, 2004; Kutch and Valero-Cuevas, 2012; Ting and Macpherson, 2005; Torres-Oviedo and Ting, 2007; Tresch et al., 2006, 1999). Conceptually, AutoLFADS approaches the problem in a different way by using an RNN to simultaneously exploit both spatial and temporal regularities. AutoLFADS builds on advances in training RNNs (e.g., using sequential autoencoders) that have enabled unsupervised characterization of complex dynamical systems, and large-scale optimization approaches that allow neural networks to be applied out-of-the-box to a wide variety of data. Capitalizing on these advances allows a great deal of flexibility in estimating the regularities underlying muscle coordination. For example, the study approach does not require an explicit estimate of dimensionality, does not restrict the modelled muscle activity to the span of a fixed time-varying basis set (Alessandro et al., 2012; d'Avella et al., 2003), does not assume linear interactions in the generative model underlying the observed muscle activations, and has an appropriate (and easily adapted) noise model to relate inferred activations to observed data. This flexibility enabled us to robustly estimate neural drive to muscles across a variety of conditions: different phases of the same behavior (stance vs. swing), different animals (rat vs. monkey), behaviors with distinct dynamics (locomotion vs. isometric contractions), and different time scales (100's of ms vs. several seconds). AutoLFADS can therefore be used by investigators studying motor control in a wide range of experimental conditions, behaviors, and organisms.

### Limitations

EMG is susceptible to artifacts (e.g., powerline noise, movement artifact, cross-talk, etc.), therefore any method that aims to extract muscle activation from EMG necessitates intelligent choice of preprocessing to mitigate the potential deleterious effects of these noise sources (see Methods). As a powerful deep learning framework that is capable of modeling nonlinear signals, AutoLFADS has a specific sensitivity to even a small number of high-amplitude artifacts that can skew model training, since producing estimates that capture these artifacts can actually lower the reconstruction cost that the model is trying to minimize. The study implementation of data augmentation strategies during training helped mitigate the sensitivity of AutoLFADS to spurious high-magnitude artifacts. In particular, in the current datasets, muscle activation estimates from AutoLFADS were more informative about behavior and brain activity than standard approaches. This high performance suggests that noise sources in our multi-muscle EMG recordings did not critically limit the method's ability to infer estimates of muscle activation. However, these factors may be more limiting in recordings with substantial noise. Future studies may be able to explore additional methods, such as regularization strategies or down-weighting reconstruction costs for high-magnitude signals, to further mitigate the effects of artifacts.

### Potential Future Applications

The applications of AutoLFADS may extend beyond scientific discovery to improved clinical devices that require accurate measurement of muscle activation, such as bionic exoskeletons for stroke patients or myoelectric prostheses for control of assistive devices. Current state of the art myoelectric prostheses use supervised classification to translate EMGs into control signals (Ameri et al., 2018; Hargrove et al., 2017; Vu et al., 2020). AutoLFADS could complement current methods through unsupervised pre-training to estimate muscle activation from EMG signals before they are input to classification algorithms. The idea of unsupervised pre-training has gained popularity in the field of natural language processing (Qiu et al., 2020). In these applications, neural networks that are pre-trained on large repositories of unlabeled text data uncover more informative representations than do standard feature selection approaches, which improves accuracy and generalization in subsequent tasks. Similar advantages have been demonstrated for neural population activity by unsupervised pre-training using LFADS, where the model's inferred firing rates enable decoding of behavioral correlates with substantially higher accuracy and better generalization to held-out data than standard methods of neural data preprocessing (Keshtkaran et al., 2021; Pandarinath et al., 2018).

Designing AutoLFADS models that require minimal or no network re-training to work across subjects could help drive translation to clinical applications. Another area of deep learning research—termed transfer learning—has produced methods that transfer knowledge from neural networks trained on one dataset (termed the source domain) to inform application to different data (termed the target domain) (Zhuang et al., 2020). Developing subject-independent AutoLFADS models using transfer learning approaches would reduce the need for subject-specific data collection that may be difficult to perform outside of controlled, clinical environments. Subject-independent models of muscle coordination may also benefit brain-controlled functional electrical stimulation (FES) systems that rely on mapping motor cortical activity onto stimulation commands to restore muscle function (Ajiboye et al., 2017; Ethier et al., 2012). Components of subject-independent AutoLFADS models trained on muscle activity from healthy patients, could be adapted for use in decoder models that aim to map simultaneous recordings of motor cortical activity onto predictions of muscle activation for patients with paralysis. These approaches may provide a neural network solution to decode muscle activation commands from brain activity more accurately, serving to improve the estimates of stimulation commands for FES implementations. Combining AutoLFADS with transfer learning methods could also enable the development of new myoelectric prostheses that infer the function of missing muscles by leveraging information about the coordinated activation of remaining muscles.

There are several aspects of the AutoLFADS approach that might provide additional insights into the neural control of movement. First, AutoLFADS also provides an estimate of the time-varying input to the underlying dynamical system (i.e., u[t]). The input represents an unpredictable change to the underlying muscle activation state, which likely reflects commands from upstream brain areas that converge with sensory feedback at the level of the spinal cord, as well as reflexive inputs. Analyzing u[t] in cases where we also have simultaneous recordings of relevant upstream brain areas may allow us to more precisely distinguish the contributions of different upstream sources to muscle coordination. Second, AutoLFADS may also be useful for studying complex, naturalistic behavior. In this study we applied AutoLFADS to activity from two relatively simple, yet substantially different motor tasks, each with stereotypic structure. However, because the method is applied to windows of activity without regard to behavior, it does not require repeated, similar trials or alignment to behavioral events. Even with this unsupervised approach, AutoLFADS preserved information about behavior (kinematics and forces) with higher fidelity than standard filtering to preserve behaviorally-relevant features. As supervision becomes increasingly challenging during behaviors that are less constrained or difficult to carefully monitor (e.g., free cage movements), unsupervised approaches like AutoLFADS may be necessary to extract precise estimates of muscle activity. AutoLFADS may therefore have the potential to overcome traditional barriers to studying neural control of movement by allowing investigators to study muscle coordination during unconstrained, ethologically-relevant behaviors rather than the constrained, artificial behaviors often used in traditional studies.

### Conclusion

The study demonstrated that AutoLFADS is a powerful tool to estimate muscle activation from EMG activity, complementing the analysis of cortical activity to which it has previously been applied. In the study tests, AutoLFADS outperformed standard filtering approaches in estimating muscle activation. In particular, the AutoLFADS-inferred muscle activation commands were substantially more informative about both behavior and brain activity, all in an unsupervised manner that did not require manual tuning of hyperparameters. Further, we observed robust performance across data from two different species (rat and monkey) performing different behaviors that elicited muscle activations at varying timescales and with very different dynamics. The lack of prior assumptions about behaviorally-relevant timescales is critical, as there is growing evidence that precise timing plays an important role in motor control, and that high-frequency features may be important for understanding the structure of motor commands (Sober et al., 2018; Srivastava et al., 2017). The study demonstrated that AutoLFADS is capable of extracting high-frequency features underlying muscle commands—such as the muscle activity related to foot off during locomotion, or the oscillations uncovered during isometric force contraction—that are difficult to analyze using conventional filtering. By improving our ability to estimate short-timescale features in muscle activation, AutoLFADS may serve to increase the precision with which EMG signals are analyzed when studying motor control or translating them to control applications.

### Example of a Computing System

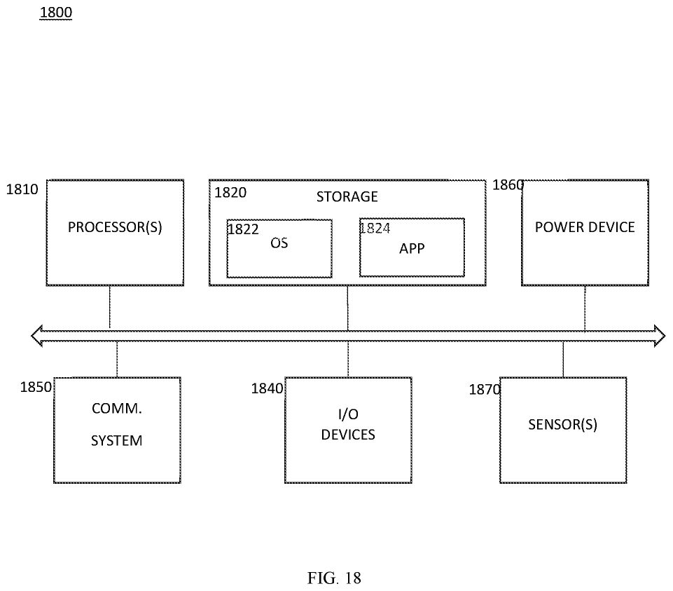

FIG. 18 depicts a block diagram of an example computing system 1800 for implementing certain embodiments. For example, in some aspects, the computer system 1800 may include computing systems associated with a device (e.g., the device 110) performing one or more processes (e.g., FIGS. 2, 3, and 7-10) disclosed herein. The block diagram illustrates some electronic components or subsystems of the computing system. The computing system 1800 depicted in FIG. 18 is merely an example and is not intended to unduly limit the scope of inventive embodiments recited in the claims. One of ordinary skill in the art would recognize many possible variations, alternatives, and modifications. For example, in some implementations, the computing system 1800 may have more or fewer subsystems than those shown in FIG. 18, may combine two or more subsystems, or may have a different configuration or arrangement of subsystems.

In the example shown in FIG. 18, the computing system 1800 may include one or more processing units 1810 and storage 1820. The processing units 1810 may be configured to execute instructions for performing various operations, and can include, for example, a microcontroller, a general-purpose processor, or a microprocessor suitable for implementation within a portable electronic device, such as a Raspberry Pi. The processing units 1810 may be communicatively coupled with a plurality of components within the computing system 1800. For example, the processing units 1810 may communicate with other components across a bus. The bus may be any subsystem adapted to transfer data within the computing system 1800. The bus may include a plurality of computer buses and additional circuitry to transfer data.

In some embodiments, the processing units 1810 may be coupled to the storage 1820. In some embodiments, the storage 1820 may offer both short-term and long-term storage and may be divided into several units. The storage 1820 may be volatile, such as static random access memory (SRAM) and/or dynamic random access memory (DRAM), and/or non-volatile, such as read-only memory (ROM), flash memory, and the like. Furthermore, the storage 1820 may include removable storage devices, such as secure digital (SD) cards. The storage 1820 may provide storage of computer readable instructions, data structures, program modules, audio recordings, image files, video recordings, and other data for the computing system 1800. In some embodiments, the storage 1820 may be distributed into different hardware modules. A set of instructions and/or code might be stored on the storage 1820. The instructions might take the form of executable code that may be executable by the computing system 1800, and/or might take the form of source and/or installable code, which, upon compilation and/or installation on the computing system 1800 (e.g., using any of a variety of generally available compilers, installation programs, compression/decompression utilities, and the like), may take the form of executable code.

In some embodiments, the storage 1820 may store a plurality of application modules 1824, which may include any number of applications, such as applications for controlling input/output (I/O) devices 1840 (e.g., sensor(s) (e.g., sensor(s) 1870, other sensor(s), etc.)), a switch, a camera, a microphone or audio recorder, a speaker, a media player, a display device, target device 130, etc.). The application modules 1824 may include particular instructions to be executed by the processing units 1810. In some embodiments, certain applications, or parts of the application modules 1824 may be executable by other hardware modules, such as a communication subsystem 1850. In certain embodiments, the storage 1820 may additionally include secure memory, which may include additional security controls to prevent copying or other unauthorized access to secure information.

In some embodiments, the storage 1820 may include an operating system 1822 loaded therein, such as an Android operating system or any other operating system suitable for mobile devices or portable devices. The operating system 1822 may be operable to initiate the execution of the instructions provided by the application modules 1824 and/or manage other hardware modules as well as interfaces with a communication subsystem 1850 which may include one or more wireless or wired transceivers. The operating system 1822 may be adapted to perform other operations across the components of the computing system 1800 including threading, resource management, data storage control, and other similar functionality.

The communication subsystem 1850 may include, for example, an infrared communication device, a wireless communication device and/or chipset (such as a Bluetooth® device, an IEEE 802.11 (Wi-Fi) device, a WiMax device, cellular communication facilities, and the like), NFC, ZigBee, and/or similar communication interfaces. The computing system 1800 may include one or more antennas (not shown in FIG. 18) for wireless communication as part of the communication subsystem 1850 or as a separate component coupled to any portion of the system.

Depending on desired functionality, the communication subsystem 1850 may include separate transceivers to communicate with base transceiver stations and other wireless devices and access points, which may include communicating with different data networks and/or network types, such as wireless wide-area networks (WWANs), WLANs, or wireless personal area networks (WPANs). A WWAN may be, for example, a WiMax (IEEE 802.9) network. A WLAN may be, for example, an IEEE 802.11x network. A WPAN may be, for example, a Bluetooth network, an IEEE 802.15x, or some other types of network. The techniques described herein may also be used for any combination of WWAN, WLAN, and/or WPAN. In some embodiments, the communications subsystem 1850 may include wired communication devices, such as Universal Serial Bus (USB) devices, Universal Asynchronous Receiver/Transmitter (UART) devices, Ethernet devices, and the like. The communications subsystem 1850 may permit data to be exchanged with a network, other computing systems, and/or any other devices described herein. The communication subsystem 1850 may include a means for transmitting or receiving data, such as identifiers of portable goal tracking devices, position data, a geographic map, a heat map, photos, or videos, using antennas and wireless links. The communication subsystem 1850, the processing units 1810, and the storage 1820 may together comprise at least a part of one or more of a means for performing some functions disclosed herein.

The computing system 1800 may include one or more I/O devices 1840, such as sensors 1870, a switch, a camera, a microphone or audio recorder, a communication port, or the like. For example, the I/O devices 1840 may include one or more touch sensors or button sensors associated with the buttons. The touch sensors or button sensors may include, for example, a mechanical switch or a capacitive sensor that can sense the touching or pressing of a button.

In some embodiments, the I/O devices 1840 may include a microphone or audio recorder that may be used to record an audio message. The microphone and audio recorder may include, for example, a condenser or capacitive microphone using silicon diaphragms, a piezoelectric acoustic sensor, or an electret microphone. In some embodiments, the microphone and audio recorder may be a voice-activated device. In some embodiments, the microphone and audio recorder may record an audio clip in a digital format, such as MP3, WAV, WMA, DSS, etc. The recorded audio files may be saved to the storage 1820 or may be sent to the one or more network servers through the communication subsystem 1850.

In some embodiments, the I/O devices 1840 may include a location tracking device, such as a global positioning system (GPS) receiver. In some embodiments, the I/O devices 1840 may include a wired communication port, such as a micro-USB, Lightning, or Thunderbolt transceiver.

The I/O devices 1840 may also include, for example, a speaker, a media player, a display device, a communication port, or the like. For example, the I/O devices 1840 may include a display device, such as an LED or LCD display and the corresponding driver circuit. The I/O devices 1840 may include a text, audio, or video player that may display a text message, play an audio clip, or display a video clip.

The computing system 1800 may include a power device 1860, such as a rechargeable battery for providing electrical power to other circuits on the computing system 1800. The rechargeable battery may include, for example, one or more alkaline batteries, lead-acid batteries, lithium-ion batteries, zinc-carbon batteries, and NiCd or NiMH batteries. The computing system 1800 may also include a battery charger for charging the rechargeable battery. In some embodiments, the battery charger may include a wireless charging antenna that may support, for example, one of Qi, Power Matters Association (PMA), or Association for Wireless Power (A4WP) standard, and may operate at different frequencies. In some embodiments, the battery charger may include a hard-wired connector, such as, for example, a micro-USB or Lightning® connector, for charging the rechargeable battery using a hard-wired connection. The power device 1860 may also include some power management integrated circuits, power regulators, power convertors, and the like.

In some embodiments, the computing system 1800 may include one or more sensors 1870. The sensors 1870 may include, for example, the neuromuscular sensors 120 as described above.

The computing system 1800 may be implemented in many different ways. In some embodiments, the different components of the computing system 1800 described above may be integrated to a same printed circuit board. In some embodiments, the different components of the computing system 1800 described above may be placed in different physical locations and interconnected by, for example, electrical wires. The computing system 1800 may be implemented in various physical forms and may have various external appearances. The components of computing system 1800 may be positioned based on the specific physical form.

The methods, systems, and devices discussed above are examples. Various embodiments may omit, substitute, or add various procedures or components as appropriate. For instance, in alternative configurations, the methods described may be performed in an order different from that described, and/or various stages may be added, omitted, and/or combined. Also, features described with respect to certain embodiments may be combined in various other embodiments. Different aspects and elements of the embodiments may be combined in a similar manner. Also, technology evolves and, thus, many of the elements are examples that do not limit the scope of the disclosure to those specific examples.

The foregoing method descriptions and the process flow diagrams are provided merely as illustrative examples and are not intended to require or imply that the operations of various embodiments must be performed in the order presented. As will be appreciated by one of skill in the art the order of operations in the foregoing embodiments may be performed in any order. Words such as “thereafter,” “then,” “next,” etc. are not intended to limit the order of the operations; these words are simply used to guide the reader through the description of the methods. Further, any reference to claim elements in the singular, for example, using the articles “a,” “an” or “the” is not to be construed as limiting the element to the singular.

While the terms “first” and “second” are used herein to describe data transmission associated with a subscription and data receiving associated with a different subscription, such identifiers are merely for convenience and are not meant to limit various embodiments to a particular order, sequence, type of network or carrier.

Various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such embodiment decisions should not be interpreted as causing a departure from the scope of the claims.

The hardware used to implement various illustrative logics, logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing systems, (e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In one or more example embodiments, the functions described may be implemented in hardware, software, firmware, or any combination thereof. If implemented in software, the functions may be stored as one or more instructions or code on a non-transitory computer readable medium or non-transitory processor-readable medium. The operations of a method or algorithm disclosed herein may be embodied in a processor-executable software module, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable medium and/or computer-readable medium, which may be incorporated into a computer program product.

Those of skill in the art will appreciate that information and signals used to communicate the messages described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Terms, “and” and “or” as used herein, may include a variety of meanings that also is expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, AB, AC, BC, AA, ABC, AAB, AABBCCC, and the like.

Further, while certain embodiments have been described using a particular combination of hardware and software, it should be recognized that other combinations of hardware and software are also possible. Certain embodiments may be implemented only in hardware, or only in software, or using combinations thereof. In one example, software may be implemented with a computer program product containing computer program code or instructions executable by one or more processors for performing any or all of the steps, operations, or processes described in this disclosure, where the computer program may be stored on a non-transitory computer readable medium. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques, including, but not limited to, conventional techniques for inter-process communications, and different pairs of processes may use different techniques, or the same pair of processes may use different techniques at different times.

### REFERENCES

Ajiboye A B, Willett F R, Young D R, Memberg W D, Murphy B A, Miller J P, Walter B L, Sweet J A, Hoyen H A, Keith M W, Peckham P H, Simeral J D, Donoghue J P, Hochberg L R, Kirsch R F. 2017. Restoration of reaching and grasping movements through brain-controlled muscle stimulation in a person with tetraplegia: a proof-of-concept demonstration. The Lancet 389:1821-1830. doi: 10.1016/S0140-6736 (17) 30601-3
Alessandro C, Barroso F O, Prashara A, Tentler D P, Yeh H-Y, Tresch M C. 2020. Coordination amongst quadriceps muscles suggests neural regulation of internal joint stresses, not simplification of task Proc Natl Acad performance. Sci 117:8135-8142. doi: 10.1073/pnas.1916578117
Alessandro C, Carbajal J P, d'Avella A. 2012. Synthesis and Adaptation of Effective Motor Synergies for the Solution of Reaching Tasks In: Ziemke T, Balkenius C, Hallam J, editors. From Animals to Animats 12, Lecture Notes in Computer Science. Berlin, Heidelberg: Springer. pp. 33-43. doi: 10.1007/978-3-642-33093-3_4
Alessandro C, Rellinger B A, Barroso F O, Tresch M C. 2018. Adaptation after vastus lateralis denervation in rats demonstrates neural regulation of joint stresses and strains. eLife 7: e38215. doi: 10.7554/eLife.38215
Ameri A, Akhaee M A, Scheme E, Englehart K. 2018. Real-time, simultaneous myoelectric control using a convolutional neural network. PLOS ONE 13: e0203835. doi: 10.1371/journal.pone.0203835
Bauman J M, Chang Y-H. 2010. High-speed X-ray video demonstrates significant skin movement errors with standard optical kinematics during rat locomotion. J Neurosci Methods 186:18-24. doi: 10.1016/j.jneumeth.2009.10.017
Boonstra T W, Farmer S F, Breakspear M. 2016. Using Computational Neuroscience to Define Common Input to Spinal Motor Neurons. Front Hum Neurosci 10. doi: 10.3389/fnhum.2016.00313
Clancy E A, Bouchard S, Rancourt D. 2001. Estimation and application of EMG amplitude during dynamic contractions. IEEE Eng Med Biol Mag 20:47-54. doi: 10.1109/51.982275
d'Avella A, Saltiel P, Bizzi E. 2003. Combinations of muscle synergies in the construction of a natural motor behavior. Nat Neurosci 6:300-308. doi: 10.1038/nn1010
D'Alessio T, Conforto S. 2001. Extraction of the envelope from surface EMG signals. IEEE Eng Med Biol Mag 20:55-61. doi: 10.1109/51.982276
De Luca C J, Adam A, Wotiz R, Gilmore L D, Nawab S H. 2006. Decomposition of Surface EMG Signals. J Neurophysiol 96:1646-1657. doi: 10.1152/jn.00009.2006
De Luca C J, Erim Z. 1994. Common drive of motor units in regulation of muscle force. Trends Neurosci 17:299-305. doi: 10.1016/0166-2236 (94) 90064-7
Del Vecchio A, Holobar A, Falla D, Felici F, Enoka R M, Farina D. 2020. Tutorial: Analysis of motor unit discharge characteristics from high-density surface EMG signals. J Electromyogr Kinesiol 53:102426. doi: 10.1016/j.jelekin.2020.102426
Dideriksen J L, Farina D. 2019. Amplitude cancellation influences the association between frequency components in the neural drive to muscle and the rectified EMG signal. PLOS Comput Biol 15: e1006985. doi: 10.1371/journal.pcbi.1006985
Dyson M, Barnes J, Nazarpour K. 2017. Abstract myoelectric control with EMG drive estimated using linear, kurtosis and Bayesian filtering 2017 8th International IEEE/EMBS Conference on Neural Engineering (NER). Presented at the 2017 8th International IEEE/EMBS Conference on Neural Engineering (NER). pp. 54-57. doi: 10.1109/NER.2017.8008290
Ethier C, Oby E R, Bauman M J, Miller L E. 2012. Restoration of grasp following paralysis through brain-controlled of muscles. Nature 485:368-371. stimulation doi: 10.1038/nature10987
Farina D, Merletti R, Enoka R M. 2014. The extraction of neural strategies from the surface EMG: an update. J Appl Physiol Bethesda Md 1985 117:1215-1230. doi: 10.1152/japplphysiol.00162.2014
Farina D, Merletti R, Enoka R M. 2004. The extraction of neural strategies from the surface EMG. J Appl Physiol 96:1486-1495. doi: 10.1152/japplphysiol.01070.2003
Farina D, Negro F. 2015. Common Synaptic Input to Motor Neurons, Motor Unit Synchronization, and Force Control. Exerc Sport Sci Rev 43:23-33. doi: 10.1249/JES.0000000000000032
Farina D, Negro F, Muceli S, Enoka R M. 2016. Principles of Motor Unit Physiology Evolve With Advances in Technology. Physiology 31:83-94. doi: 10.1152/physiol.00040.2015
Filipe V M, Pereira J E, Costa L M, Mauricio A C, Couto P A, Melo-Pinto P, Varejão ASP. 2006. Effect of skin movement on the analysis of hindlimb kinematics during treadmill locomotion in rats. J Neurosci Methods 153:55-61. doi: 10.1016/j.jneumeth.2005.10.006
Gallego J A, Perich M G, Chowdhury R H, Solla S A, Miller L E. 2020. Long-term stability of cortical population dynamics underlying consistent behavior. Nat Neurosci 23:260-270. doi: 10.1038/s41593-019-0555-4
Hargrove L J, Miller L A, Turner K, Kuiken T A. 2017. Myoelectric Pattern Recognition Outperforms Direct Control for Transhumeral Amputees with Targeted Muscle Reinnervation: A Randomized Clinical Trial. Sci Rep 7:13840. doi: 10.1038/s41598-017-14386-w
Hart C B, Giszter S F. 2004. Modular Premotor Drives and Unit Bursts as Primitives for Frog Motor Behaviors. J Neurosci 24:5269-5282. doi: 10.1523/JNEUROSCI.5626-03.2004
Hofmann D, Jiang N, Vujaklija I, Farina D. 2016. Bayesian Filtering of Surface EMG for Accurate Simultaneous and Proportional Prosthetic Control. IEEE Trans Neural Syst Rehabil Eng 24:1333-1341. doi: 10.1109/TNSRE.2015.2501979
Hogan N, Mann R W. 1980. Myoelectric Signal Processing: Optimal Estimation Applied to Electromyography-Part I: Derivation of the Optimal Myoprocessor. IEEE Trans Biomed Eng BME-27:382-395. doi: 10.1109/TBME.1980.326652
Jaderberg M, Dalibard V, Osindero S, Czarnecki W M, Donahue J, Razavi A, Vinyals O, Green T, Dunning I, Simonyan K, Fernando C, Kavukcuoglu K. 2017. Population Based Training of Neural Networks. ArXiv171109846 Cs.
Keshtkaran M R, Pandarinath C. 2019. Enabling hyperparameter optimization in sequential autoencoders for spiking neural data. Adv Neural Inf Process Syst 32:15937-15947.
Keshtkaran M R, Sedler A R, Chowdhury R H, Tandon R, Basrai D, Nguyen S L, Sohn H, Jazayeri M, Miller L E, Pandarinath C. 2021. A large-scale neural network training framework for generalized estimation of single-trial population dynamics. bioRxiv 2021.01.13.426570. doi: 10.1101/2021.01.13.426570
Keshtkaran M R, Yang Z. 2014. A fast, robust algorithm for power line interference cancellation in neural recording. J Neural Eng 11:026017. doi: 10.1088/1741-2560/11/2/026017
Kingma D P, Welling M. 2014. Auto-Encoding Variational Bayes. ArXiv13126114 Cs Stat.
Kutch J J, Valero-Cuevas F J. 2012. Challenges and New Approaches to Proving the Existence of Muscle Synergies of Neural Origin. PLOS Comput Biol 8: e1002434. doi: 10.1371/journal.pcbi.1002434
Nasr A, Bell S, He J, Whittaker R L, Jiang N, Dickerson C R, McPhee J. 2021. MuscleNET: mapping electromyography to kinematic and dynamic biomechanical variables by machine learning. J Neural Eng 18: 0460d3. doi: 10.1088/1741-2552/acladc
Nazarpour K, Al-Timemy A H, Bugmann G, Jackson A. 2013. A note on the probability distribution function of the surface electromyogram signal. Brain Res Bull 90:88-91. doi: 10.1016/j.brainresbull.2012.09.012
Negro F, Keenan K, Farina D. 2015. Power spectrum of the rectified EMG: when and why is rectification beneficial for identifying neural connectivity? J Neural Eng 12:036008. doi: 10.1088/1741-2560/12/3/036008
Pandarinath C, O'Shea D J, Collins J, Jozefowicz R, Stavisky S D, Kao J C, Trautmann E M, Kaufman M T, Ryu S I, Hochberg L R, Henderson J M, Shenoy K V, Abbott L F, Sussillo D. 2018. Inferring single-trial neural population dynamics using sequential auto-encoders. Nat Methods 15:805-815. doi: 10.1038/s41592-018-0109-9
Qiu X, Sun T, Xu Y, Shao Y, Dai N, Huang X. 2020. Pre-trained Models for Natural Language Processing: A Survey. ArXiv200308271 Cs.
Sanger T D. 2007. Bayesian Filtering of Myoelectric Signals. J Neurophysiol 97:1839-1845. doi: 10.1152/jn.00936.2006
Sober S J, Sponberg S, Nemenman I, Ting L H. 2018. Millisecond Spike Timing Codes for Motor Control. Trends Neurosci, Special Issue: Time in the Brain 41:644-648. doi: 10.1016/j.tins.2018.08.010
Srivastava K H, Holmes C M, Vellema M, Pack A R, Elemans C P H, Nemenman I, Sober S J. 2017. Motor control by precisely timed spike patterns. Proc Natl Acad Sci 114:1171-1176. doi: 10.1073/pnas. 1611734114
Sussillo D, Jozefowicz R, Abbott L F, Pandarinath C. 2016. LFADS-Latent Factor Analysis via Dynamical Systems. ArXiv160806315 Cs Q-Bio Stat.
Ting L H, Macpherson J M. 2005. A Limited Set of Muscle Synergies for Force Control During a Postural Task. J Neurophysiol 93:609-613. doi: 10.1152/jn.00681.2004
Torres-Oviedo G, Ting L H. 2007. Muscle Synergies Characterizing Human Postural Responses. J Neurophysiol 98:2144-2156. doi: 10.1152/jn.01360.2006
Tresch M C, Cheung V C K, d'Avella A. 2006. Matrix Factorization Algorithms for the Identification of Muscle Synergies: Evaluation on Simulated and Experimental Data Sets. J Neurophysiol 95:2199-2212. doi: 10.1152/jn.00222.2005
Tresch M C, Jarc A. 2009. The case for and against muscle synergies. Curr Opin Neurobiol, Motor systems. Neurology of behaviour 19:601-607. doi: 10.1016/j.conb.2009.09.002
Tresch M C, Saltiel P, Bizzi E. 1999. The construction of movement by the spinal cord. Nat Neurosci 2:162-167. doi: 10.1038/5721
Vu P P, Vaskov A K, Irwin Z T, Henning P T, Lueders D R, Laidlaw A T, Davis A J, Nu C S, Gates D H, Gillespie R B, Kemp S W P, Kung T A, Chestek C A, Cederna P S. 2020. A regenerative peripheral nerve interface allows real-time control of an artificial hand in upper limb amputees. Sci Transl Med 12. doi: 10.1126/scitranslmed.aay2857
Williams A H, Poole B, Maheswaranathan N, Dhawale A K, Fisher T, Wilson C D, Brann D H, Trautmann E M, Ryu S, Shusterman R, Rinberg D, Ölveczky B P, Shenoy K V, Ganguli S. 2020. Discovering Precise Temporal Patterns in Large-Scale Neural Recordings through Robust and Interpretable Time Warping. Neuron 105:246-259.e8. doi: 10.1016/j.neuron.2019.10.020
Zhuang F, Qi Z, Duan K, Xi D, Zhu Y, Zhu H, Xiong H, He Q. 2020. A Comprehensive Survey on Transfer Learning. ArXiv191102685 Cs Stat.

The disclosures of each and every publication cited herein are hereby incorporated herein by reference in their entirety.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

1. A computer-implemented method, comprising:
receiving, via one or more channels of one or more neuromuscular sensors, neuromuscular data for a preset window from each channel; and
training at least one or more networks using the first augmented data and the second augmented data, the one or more networks including one or more representation learning neural networks, wherein training the one or more representation learning neural networks includes:
randomly generating a first augment and a second augment different from the first augment for each channel;
augmenting the neuromuscular data of each channel by applying the first augment and the second augment to the neuromuscular data of respective channel to generate a first augmented data and a second augmented data for the respective channel;
processing at least the first augmented data for each channel through at least a first representation learning neural network to determine a first latent representation of one or more neuromuscular activation state variables for the respective channel;
determining a reconstruction cost using the first latent representation and the second augmented data for each channel; and
updating the first representation learning neural network based on the reconstruction cost.

2. The method according to claim 1, wherein each augment corresponds to a temporal shift, each temporal shift having an integer value.

3. The method according to claim 2, wherein:
the augmenting the neuromuscular data for each channel includes moving the neuromuscular data forward or backward temporally within the preset window based on the integer value of the respective temporal shift.

4. The method according to claim 3, wherein the one or more networks includes a second representation neural learning network, the method further comprising:
processing the second augmented data for each channel through a second representation learning neural network to determine a second latent representation of one or more neuromuscular activation state variables for the preset window of time;
wherein the reconstruction cost is determined using the first latent representation and the second latent representation; and
updating the first representation learning neural network and/or the second representation learning neural network based on the reconstruction cost.

5. The method according to claim 4, wherein the one or more networks includes a projector network, the method further comprising:
processing the first latent representation of one or more neuromuscular activation state variables for the preset window of time through a projector network to generate a first projected latent representation;
wherein the reconstruction cost is determined between the first projected latent representation and the second latent representation; and
updating the first representation learning neural network and/or the second representation learning neural network based on the reconstruction cost.

6. The method according to claim 1, wherein the one or more representation learning neural networks includes one or more of autoencoder neural networks, one or more of transformer neural networks and/or one or more of linear transformation networks.

7. The method according to claim 1, wherein each augment includes a temporal value, a magnitude value, a randomly generated temporal order, and/or a randomly generated binary mask.

8. A system, comprising:
one or more processors;
one or more neuromuscular sensors coupled to the processor, each neuromuscular sensor including one or more channels; and
a memory having stored thereon computer-executable instructions which are executable by the one or more processors to cause the computing system to perform at least the following:
receiving, via one or more channels of one or more neuromuscular sensors, neuromuscular data for a preset window from each channel; and
training at least one or more networks using the first augmented data and the second augmented data, the one or more networks including one or more representation learning neural networks, wherein the training the one or more representation learning neural networks includes:
randomly generating a first augment and a second augment different from the first augment for each channel;
augmenting the neuromuscular data of each channel by applying the first augment and the second augment to the neuromuscular data of respective channel to generate a first augmented data and a second augmented data for the respective channel;
processing at least the first augmented data for each channel through at least a first representation learning neural network to determine a first latent representation of one or more neuromuscular activation state variables for the respective channel;
determining a reconstruction cost using the first latent representation and the second augmented data for each channel; and
updating the first representation learning neural network based on the reconstruction cost.

9. The system according to claim 8, wherein each augment corresponds to a temporal shift, each temporal shift having an integer value.

10. The system according to claim 9, wherein:
the augmenting the neuromuscular data for each channel includes moving the neuromuscular data forward or backward temporally within the preset window based on the integer value of the respective temporal shift.

11. The system according to claim 10, wherein:
the one or more networks includes a first representation learning neural network; and the one or more processors are further configured to cause the computing system to perform at least the following:
processing the second augmented data for each channel through a second representation learning neural network to determine a second latent representation of one or more neuromuscular activation state variables for the preset window of time;
wherein the reconstruction cost is determined using the first latent representation and the second latent representation; and
updating the first representation learning neural network and/or the second representation learning neural network based on the reconstruction cost.

12. The system according to claim 11, wherein:
the one or more networks includes a projector network; and the one or more processors are further configured to cause the computing system to perform at least the following:
processing the first latent representation of one or more neuromuscular activation state variables for the preset window of time through a projector network to generate a first projected latent representation;
wherein the reconstruction cost is determined between the first projected latent representation and the second latent representation; and
updating the first representation learning neural network and/or the second representation learning neural network based on the reconstruction cost.

13. The system according to claim 1, wherein the one or more representation learning neural networks includes one or more of autoencoder neural networks, one or more of transformer neural networks and/or one or more of linear transformation networks.

14. The system according to claim 1, wherein each augment includes a temporal value, a magnitude value, a randomly generated temporal order, and/or a randomly generated binary mask.

15. A non-transitory computer-readable storage medium comprising one or more computer-executable instructions, that when executed by one or more processors of a computing device, cause the computing device to perform at least the following:
receiving, via one or more channels of one or more neuromuscular sensors, neuromuscular data for a preset window from each channel; and
training at least one or more networks using the first augmented data and the second augmented data, the one or more networks including one or more representation learning neural networks, wherein the training the one or more representation learning neural networks includes:
randomly generating a first augment and a second augment different from the first augment for each channel;
augmenting the neuromuscular data of each channel by applying the first augment and the second augment to the neuromuscular data of respective channel to generate a first augmented data and a second augmented data for the respective channel;
processing at least the first augmented data for each channel through at least a first representation learning neural network to determine a first latent representation of one or more neuromuscular activation state variables for the respective channel;
determining a reconstruction cost using the first latent representation and the second augmented data for each channel; and
updating the first representation learning neural network based on the reconstruction cost.

16. The medium according to claim 15, wherein each augment corresponds to a temporal shift, each temporal shift having an integer value.

17. The medium according to claim 16, wherein:
the augmenting the neuromuscular data for each channel includes moving the neuromuscular data forward or backward temporally within the preset window based on the integer value of the respective temporal shift.

18. The medium according to claim 17, wherein:
the one or more networks includes a first representation learning neural network; and the one or more processors are further configured to cause the computing system to perform at least the following:
processing the second augmented data for each channel through a second representation learning neural network to determine a second latent representation of one or more neuromuscular activation state variables for the preset window of time;
wherein the reconstruction cost is determined using the first latent representation and the second latent representation; and
updating the first representation learning neural network and/or the second representation learning neural network based on the reconstruction cost.

19. The medium according to claim 18, wherein:
the one or more networks includes a projector network; and the one or more processors are further configured to cause the computing system to perform at least the following:
processing the first latent representation of one or more neuromuscular activation state variables for the preset window of time through a projector network to generate a first projected latent representation;
wherein the reconstruction cost is determined between the first projected latent representation and the second latent representation; and
updating the first representation learning neural network and/or the second representation learning neural network based on the reconstruction cost.

20. The medium according to claim 15, wherein the one or more representation learning neural networks includes one or more of autoencoder neural networks, one or more of transformer neural networks and/or one or more of linear transformation networks.
